(19) United States
(12) Patent Application Publication
(10) Pub. No.: US 2023/0404934 A1
(43) Pub. Date: Dec. 21, 2023

(54) ON DEMAND AND LONG-TERM DRUG DELIVERY FROM DEGRADABLE NANOCAPSULES

(71) Applicant: University of Maryland, Baltimore County, Baltimore, MD (US)

(72) Inventors: Erin LAVIK, Ellicott City, MD (US); Sydney MENIKHEIM, Fallston, MD (US); Joshua LECKRON, Westminster, MD (US)

(21) Appl. No.: 18/299,135

(22) Filed: Apr. 12, 2023

Related U.S. Application Data
(63) Continuation of application No. PCT/US21/54902, filed on Oct. 14, 2021.
(60) Provisional application No. 63/091,353, filed on Oct. 14, 2020.

(51) Int. Cl. A61K 9/51; A61K 41/00; A61K 31/473; A61K 31/4418; A61P 27/02; A61K 9/00

(52) U.S. Cl. CPC A61K 9/5146; A61K 9/5192; A61K 41/0047; A61K 31/473; A61K 31/4418; A61P 27/02; A61K 9/0048

(57) ABSTRACT

A long-term delivery system for age-related macular degeneration (AMD) that can address the multifactorial nature of the disease. A polyurethane nanocapsule is disclosed that includes encapsulated molecules that treat AMD. The molecules can be passively delivered from the polyurethane nanocapsules over many weeks or months and/or can be tuned, non-invasively, using ultrasound to trigger the release the molecules from the polyurethane nanocapsules repeatedly in an on-demand and predictable manner. These nanocapsules have the potential to change the way diseases are treated and provide a new ultrasound-triggered drug delivery platform.

### CROSS REFERENCE TO RELATED APPLICATIONS

This application claims priority to U.S. Provisional Patent Application No. 63/091,353 filed on Oct. 14, 2020 in the name of Erin Lavik et al. and entitled “On demand and long-term drug delivery from degradable nanocapsules,” which is hereby incorporated by reference herein in its entirety.

### STATEMENT REGARDING FEDERALLY SPONSORED RESEARCH

Portions of this invention may have been financially supported by the United States Government support under a grant from the National Institutes of Health under Grant Number 1R56NS100732-01.

### FIELD

The present invention relates to polyurethane nanocapsules comprising at least one encapsulated molecule or drug, wherein the molecule or drug can be passively delivered to the environment that the polyurethane nanocapsules are administered to. Advantageously, the polyurethane nanocapsules can be tuned to deliver the molecule or drug, if needed, using noninvasive ultrasound. Further, a method of treating age-related macular degeneration (AMD) using the polyurethane nanocapsules is disclosed, wherein acriflavine and/or pirfenidone are encapsulated in polyurethane nanocapsules and administered to an eye of a subject suffering from AMD.

### BACKGROUND

On demand drug delivery has drawn a great deal of interest in recent years for the targeted treatment of glioblastomas [1], the delivery of local anesthetics [2], and the delivery of antibiotics [3]. These systems generally fall into two classes: nanomaterials that can be injected locally or systemically and can be triggered by light or ultrasound [1, 4-6] or depots that are implanted into a location and can be triggered by light or sound [3, 7-8], delivering molecules repeatedly in an on-demand fashion over long periods of time [5, 7-9].

Although both light and sound have been used as triggers, more studies have focused on ultrasound-triggered systems because of the ease with which ultrasound can be delivered to many locations in the body. Ultrasound-triggered systems offer great potential for therapeutic delivery of a range of molecules on demand. The majority of work in this area has focused on micro- and nanobubbles based on liposomes, lipoplexes, and polymerosomes [10]. These are generally coated with albumin or lipoproteins for stability in blood [11]. While the majority of microbubble work has focused on contrast agents, there are a growing number of studies evaluating delivery of therapeutics or combining therapeutics with contrast agents [112].

The majority of studies evaluating drug delivery from ultrasound-triggered systems have looked at local single timepoint delivery immediately after administration [11, 13-14]. Many of the formulations deliver their payload over minutes or hours post administration [15]. Unfortunately, the stability of these systems is a concern. Considerable advances have occurred in both long-term storage and improving the stability of liposomal formulations [16-17]. Gas encapsulated nanobubbles are not thermodynamically stable, but they can be engineered to be stable for hundreds of minutes by formulating their chemistry appropriately [18]. Microbubbles can be coated with nanoparticles which can be released from the bubbles by ultrasound [111]. This approach is similar to that of combining micro- or nanobubbles in promoting the disruption of tight junctions, followed by delivery of nanoparticles wherein the nanoparticles then deliver their contents overtime [14, 19-21]. While this is a creative, combination approach, it does not address the need for long-term delivery coupled with repeated on-demand delivery. Nanoparticle aggregates have been developed that dissociate in the presence of ultrasound [22]. These particles can deliver their contents for days and weeks, but once triggered and delivered to the site, a tumor in this case, the delivery is passive [22]. The approach leads to more particles in the tumor compared to controls but does not address the desire for an on-demand system that can be used over time.

There is a continued need for nanomaterials that are stable for long periods of time, deliver sustained amounts of drugs, and achieve repeated, on-demand delivery.

### SUMMARY

In one aspect, the present invention relates to polyurethane nanocapsules comprising a substantially spherical shell of polyurethane surrounding a core, wherein the shell comprising the polyurethane further comprises at least one molecule to be encapsulated.

In another aspect, the present invention relates to a method of making the polyurethane nanocapsules of any of the preceding claims, said method comprising:
dissolving surfactant in water and hexadecane to form a mixture;
stirring or sonicating the mixture at temperature in a range from about 35-45° C.;
adding isophorone diisocyanate (IPDI) to the stirred mixture comprising the water, surfactant, and hexadecane to form a solution;
sonicating the solution to form an emulsion;
adding a hydroxy-containing compound to the emulsion, with continued sonication; and
reacting the IPDI and the hydroxy-containing compound, with stirring, to form the polyurethane nanocapsules encapsulating the at least one molecule,
wherein the molecule to be encapsulated is added with the IPDI or the hydroxy-containing compound, depending on the solubility of the molecule to be encapsulated.

In still another aspect, the present invention relates to a method of inhibiting neovascularization in an eye of a subject in need thereof, said method comprising administering a long-term delivery system comprising polyurethane nanocapsules to the eye of said subject, wherein the polyurethane nanocapsules comprise a substantially spherical shell of polyurethane surrounding a core, wherein the shell comprising the polyurethane further comprises at least one molecule to be encapsulated, wherein the at least one molecule to be encapsulated comprises acriflavine.

In yet another aspect, the present invention relates to a method of substantially reducing or eliminating fibrosis in an eye of a subject in need thereof, said method comprising administering a long-term delivery system comprising polyurethane nanocapsules to the eye of said subject, wherein the polyurethane nanocapsules comprise a substantially spherical shell of polyurethane surrounding a core, wherein the shell comprising the polyurethane further comprises at least one molecule to be encapsulated, wherein the at least one molecule to be encapsulated comprises pirfenidone.

In another aspect, the present invention relates to a method of inhibiting neovascularization and substantially reducing or eliminating fibrosis in an eye of a subject in need thereof, said method comprising administering a combination therapy or dual delivery system comprising polyurethane nanocapsules to the eye of said subject, wherein the polyurethane nanocapsules comprise a substantially spherical shell of polyurethane surrounding a core, wherein the shell comprising the polyurethane further comprises at least one molecule to be encapsulated, wherein some portion of the polyurethane nanocapsules comprise encapsulated acriflavine and the remaining portion of the polyurethane nanocapsules comprise encapsulated pirfenidone.

In yet another aspect, the present invention relates to a method of treating wet-AMD, or preserving vision, in an eye of a subject in need thereof, said method comprising administering a long-term delivery system comprising polyurethane nanocapsules to the eye of said subject, wherein the polyurethane nanocapsules comprise a substantially spherical shell of polyurethane surrounding a core, wherein the shell comprising the polyurethane further comprises at least one molecule to be encapsulated, wherein the at least one molecule to be encapsulated is selected from acriflavine, pirfenidone, or both acriflavine and pirfenidone.

Other aspects, features and embodiments of the invention will be more fully apparent from the ensuing disclosure and appended claims.

### BRIEF DESCRIPTION OF THE FIGURES

FIG. 1 is a schematic of the synthesis method for the encapsulation of fluorescein. Note that the surfactant SDS acts as a stabilizing agent and is always present however it is not depicted fully in each step to avoid cluttering the image.

FIG. 2A is the FTIR spectra for the fluorescein-encapsulated polyurethane nanocapsules showing the critical peaks at 1550 cm⁻¹ (C—N vibration) 1637 cm⁻¹ (urea carbonyl), 1720 cm⁻¹ (C═O vibration) and 3330 cm⁻¹ (N—H vibration).

FIG. 2B is the DLS of fluorescein-encapsulated polyurethane nanocapsules showing two peaks with the larger peak, associated with the nanocapsules, of 145+/−9 nm.

FIG. 2C illustrates that the zeta potential for the fluorescein-encapsulated polyurethane nanocapsules was −60 mV+/−12 mV.

FIG. 3A is an SEM micrograph of the fluorescein-encapsulated polyurethane nanocapsules.

FIG. 3B is another SEM micrograph of the fluorescein-encapsulated polyurethane nanocapsules.

FIG. 3C is an SEM micrograph of the fluorescein-encapsulated polyurethane nanocapsules post-sonication.

FIG. 3D is a confocal image of the fluorescein-encapsulated polyurethane nanocapsules showing that the fluorescein is localized in the shells of the nanocapsules.

FIG. 3E is another confocal image of the fluorescein-encapsulated polyurethane nanocapsules showing that the fluorescein is localized in the shells of the nanocapsules.

FIG. 3F is another confocal image of the fluorescein-encapsulated polyurethane nanocapsules showing that the fluorescein is localized in the shells of the nanocapsules.

FIG. 3G is a TEM image of the fluorescein-encapsulated polyurethane nanocapsules.

FIG. 3H is another TEM image of the fluorescein-encapsulated polyurethane nanocapsules.

FIG. 4A illustrates the release of fluorescein from the fluorescein-encapsulated polyurethane nanocapsules using standard infinite sink model in PBS at 37 C. The long term delivery is replotted in FIGS. 4B and 4C as a point of comparison.

FIG. 4B illustrates the release from FIG. 4A overlaid by sonication time points. Samples were sonicated for 30 seconds at 15 minute intervals and the amount of fluorescein was measured at each time point.

FIG. 4C illustrates the release from FIG. 4A overlaid by 60 second sonication events at 15 minute intervals.

FIG. 5A is a confocal microscopy image of the acriflavine-encapsulated polyurethane nanocapsules showing the average Z-projection of the nanocapsules.

FIG. 5B is a confocal microscopy image of the acriflavine-encapsulated polyurethane nanocapsules showing a slice through the nanocapsules wherein the drug is localized in the shells of the nanocapsules. Like the fluorescein nanocapsules, acriflavine nanocapsules were far easier to visualize in clusters.

FIG. 5C is a DLS image of the acriflavine-encapsulated polyurethane nanocapsules post lyophilization having an average size of 330+/−63 nm.

FIG. 5D is a DLS image wherein the acriflavine-encapsulated polyurethane nanocapsules were sized prior to the sonication study and their average size was 295+/−33 nm.

FIG. 5E is a DLS image wherein the acriflavine-encapsulated polyurethane nanocapsules were exposed to 10 rounds of sonication for the data in FIG. 6B. Their post sonication size was 228+/−33 nm.

FIG. 6A illustrates the release curve for acriflavine-encapsulated polyurethane nanocapsules (loading: 54 ug/mg nanocapsules).

FIG. 6B illustrates the release curve overlaid with sonication release (via 20 second bursts) from acriflavine-encapsulated polyurethane nanocapsules. The green curve is the first part of the long-term release curve shown in FIG. 6A.

FIG. 6C is an image of acriflavine-encapsulated polyurethane nanocapsules in PBS in a 50 ml conical tube using the Ellex EYE CUBED ultrasound system. In the image, one can see the bottom of the 50 ml conical tube containing the nanocapsules which show bright signatures on the screen.

FIG. 6D illustrates the release of acriflavine from acriflavine-encapsulated polyurethane nanocapsules exposed to ultrasound for different times at 90 dB and 10 MHz.

FIG. 7 illustrates the release of the encapsulated molecule from the polyurethane nanocapsule upon application of ultrasound energy.

FIG. 8A is a TEM image of pirfenidone-encapsulated polyurethane nanocapsules.

FIG. 8B is another TEM image of pirfenidone-encapsulated polyurethane nanocapsules. DLS confirms nanocapsules are 245±40 nm.

FIG. 8C is an image of the pirfenidone-encapsulated polyurethane nanocapsules imaged using the Biospa imaging system. Because Pirfenidone can be excited at 310 nm and emit at 410 nm, the drug and particles can be visualized in the DAPI channel.

FIG. 8D illustrates release curves using an infinite sink release system, which shows that while PLGA-based nanoparticles of the prior art deliver the drug for 7 days, the pirfenidone-encapsulated polyurethane (PU) nanocapsules release the drug for at least 150 days with 20% of the drug released.

### DETAILED DESCRIPTION, AND PREFERRED EMBODIMENTS THEREOF

Multiple applications would benefit from an easily administered, on-demand system that combines the features of implantable depot systems with those of flexible nanoformulations. An ideal system would enable on-demand delivery over long periods of time as well as be easily administered via multiple routes. In light of this need, the present inventors developed polyurethane nanocapsules capable of long-term delivery of a molecule (e.g., a drug) as well as capable of releasing higher amounts of the molecule when triggered by ultrasound. As proof of principle, fluorescein was encapsulated in the nanocapsules described herein and both the long-term, passive delivery as well as the ultrasound-triggered release over multiple cycles investigated. In addition, an anti-angiogenic compound (acriflavine) with potential applications in age-related macular degeneration (AMD) was encapsulated in the nanocapsules described herein. It was surprisingly discovered that ultrasound triggered release of encapsulated molecules repeatedly in an on-demand manner and the amount delivered was a function of the ultrasound time. In addition, a commercially available, clinically approved clinical-grade ultrasound system typically used for ocular assessment was discovered to be capable of triggering release of encapsulated molecules.

As defined herein, “long-term therapy” is defined as at least one month, at least two months, at least three months or at least four months of therapeutic relief, alleviation of the indicated pathology with a single administration of the nanocapsules described herein. Alternatively, “long-term therapy” is defined as at least one month, at least two months, at least three months or at least four months of negligible or no further progression of the indicated pathology with a single administration of the nanocapsules described herein.

As defined herein, in “proximity of the eye” corresponds to within no more than 1 cm, preferably no more than 0.5 cm from the eye, wherein the ultrasound probe is external to the body (e.g., the point of contact is directly on an eye lid) or inserted into a nasal cavity. In one embodiment, a coupling medium is applied between the ultrasound probe and the point of contact to maximize transmission. The coupling media can include water, oils, creams, and gels, as understood by the person skilled in the art.

As defined herein, “substantially spherical” corresponds to a spherical or nearly-spherical nanocapsule. In some embodiments, the substantially spherical nanocapsule can have an average nanocapsule aspect ratio less than about 1.5, In further embodiments, the average nanocapsule aspect ratio can be less than about 1.1. As used herein, “aspect ratio” refers to the longest dimension of a nanocapsule divided by the shortest dimension of the nanocapsule. It should be appreciated by the person skilled in the art that the substantially spherical nanocapsules may look deflated following lyophilization. Further, substantially spherical allows for some flat or irregular surfaces along interface contact points.

As defined herein, “antibiotic agents” include known agents that are capable of killing or attenuating the growth of microorganisms, for example natural and synthetic penicillins and cephalosporins, sulphonamides, erythromycin, kanomycin, tetracycline, chloramphenicol, rifampicin and including gentamicin, ampicillin, benzypenicillin, benethamine penicillin, benzathine penicillin, phenethicillin, phenoxy-methyl penicillin, procaine penicillin, cloxacillin, flucloxacillin, methicillin sodium, amoxicillin, bacampicillin hydrochloride, ciclacillin, mezlocillin, pivampicillin, talampicillin hydrochloride, carfecillin sodium, piperacillin, ticarcillin, mecillinam, pirmecillinan, cefaclor, cefadroxil, cefotaxime, cefoxitin, cefsulodin sodium, ceftazidime, ceftizoxime, cefuroxime, cephalexin, cephalothin, cephamandole, cephazolin, cephradine, latamoxef disodium, aztreonam, chlortetracycline hydrochloride, clomocycline sodium, demeclocydine hydrochloride, doxycycline, lymecycline, minocycline, oxytetracycline, amikacin, framycetin sulphate, neomycin sulphate, netilmicin, tobramycin, colistin, sodium fusidate, polymyxin B sulphate, spectinomycin, vancomycin, calcium sulphaloxate, sulfametopyrazine, sulphadiazine, sulphadimidine, sulphaguanidine, sulphaurea, capreomycin, metronidazole, tinidazole, cinoxacin, ciprofloxacin, nitrofurantoin, hexamine, streptomycin, carbenicillin, colistimethate, polymyxin B, furazolidone, nalidixic acid, trimethoprim-sulfamethox-azole, clindamycin, lincomycin, cycloserine, isoniazid, ethambutol, ethionamide, pyrazinamide and the like; anti-fungal agents, for example miconazole, ketoconazole, itraconazole, fluconazole, amphotericin, flucytosine, griseofulvin, natamycin, nystatin, and the like; and anti-viral agents such as acyclovir, AZT, ddl, amantadine hydrochloride, inosine pranobex, vidarabine, and the like.

Polyurethane nanocapsules have been used extensively for self-healing dental resins and bone cements [27], essential oils [47] and enzymatically triggered drug delivery systems [48-49]. The last nanocapsules involve the incorporation of peptides into the polyurethane synthesis that are enzymatically degraded to trigger release in particular biological compartments.

Generally, the present invention uses polyurethane nanocapsules for both long-term passive delivery of molecules (e.g., drugs) as well as to deliver molecules (e.g., drugs) on-demand in a repeated fashion into an environment in proximity of the nanocapsules. In a first aspect, the present invention relates to polyurethane nanocapsules comprising a substantially spherical shell of polyurethane surrounding a core, wherein the shell comprising the polyurethane further comprises at least one molecule to be encapsulated, for example, at least one drug to be delivered in a location where the nanocapsule is positioned. The at least one molecule can be homogeneously or heterogeneously distributed throughout the substantially spherical shell of polyurethane. Some amount of the at least one molecule will be “encapsulated” in the polyurethane nanocapsule, including at least one of: being exposed on an outside surface of the shell, being exposed on an inside surface of the shell, or being contained within the polyurethane shell between the outside surface and the inside surface. In one embodiment, the core comprises air. In another embodiment, the core comprises a hydrophilic liquid such as water. In still another embodiment, the core comprises a hydrophobic liquid such as an oil. In another embodiment, the core comprises a contrast agent to assist with ultrasound analysis, for example, octafluoropropane. In yet another embodiment, the core is substantially devoid of triethylene glycol dimethyacrylate (TEGDMA). The molecule to be encapsulated includes, but is not limited to: acriflavine; pirfenidone; 4-hydroxy-TEMPO (aka TEMPOL); growth factors including, but not limited to, glial cell-derived neurotrophic factor (GDNF), brain-derived neurotrophic factor (BDNF), ciliary neurotrophic factor (CNTF), neurotrophin-3 (NT-3), and nerve growth factor (NGF); AG1478 (CAS No. 153436-53-4); methotrexate; and antibiotic agents.

The polyurethane nanocapsules comprising the at least one molecule to be encapsulated have a effective mean diameter of about 50 to about 900 nm. The nanocapsules can be tailored to create the optimum size depending on the method of administration, the amount of encapsulated molecule loaded and/or released, and the pathology to be treated. Ranges of effective mean diameters contemplated include, but are not limited to, about 50 nm to about 150 nm, about 100 nm to about 200 nm, about 150 nm to about 250 nm, about 200 nm to about 300 nm, about 300 nm to about 400 nm, about 400 nm to about 500 nm, about 500 nm to about 600 nm, about 600 nm to about 700 nm, about 700 nm to about 800 nm, about 800 nm to about 900 nm, about 50 nm to about 250 nm, about 250 nm to about 500 nm, about 500 nm to about 750 nm, or about 750 nm to about 900 nm. In one embodiment, the effective mean diameter is about 100-200 nm. In another embodiment, the effective mean diameter is about 200-300 nm. The polyurethane nanocapsules can have narrow size distribution (e.g., in a range of about 50-75 nm) or a wide size distribution (e.g., in a range of about 100-200 nm).

In one embodiment, the polyurethane nanocapsule is not PEGylated or otherwise modified. In another embodiment, the nanocapsules are PEGylated via the addition of an isocyanate functionalized polyethylene glycol (PEG) during the interfacial polymerization process [31, 54]. In one embodiment, the polyurethane nanocapsules is PEGylated to reduce aggregation of the nanocapsules.

The molecule encapsulated polyurethane nanocapsules can be prepared as readily understood by the person skilled in the art. In one embodiment, the nanocapsules are prepared following the methodology of Torini et al. [23] and Guo et al. [24]. Broadly, in one embodiment, a surfactant, such as sodium dodecyl sulfate (SDS), is dissolved in water (e.g., DI water) and hexadecane and stirred or sonicated at temperature in a range from about 35-45° C., preferably about 40° C. for approximately one hour. Isophorone diisocyanate (IPDI) is mixed with the at least one molecule to be encapsulated and water is added in dropwise to a beaker containing the water, surfactant (e.g., SDS), and hexadecane. The solution is sonicated to form the emulsion. 1,6-hexanediol (HDOH), or similar hydroxy-containing compound, is dissolved in water and added to the solution with sonication. It is then stirred overnight at 40° C. to form the polyurethane. Light exposure is preferably minimized or eliminated. The nanocapsules can be collected by centrifugation and washed before flash freezing and lyophilization.

Notably, the molecule to be encapsulated is introduced to the polyurethane synthesis based on its' solubility in IPDI versus HDOH. For example, if the molecule to be encapsulated is more soluble in IPDI in water, it is introduced with the IPDI/water mixture. If the molecule to be encapsulated is more soluble in HDOH in water and is with the HDOH/water mixture. Accordingly, in another embodiment, a surfactant, such as sodium dodecyl sulfate (SDS), is dissolved in water (e.g., DI water) and hexadecane and stirred or sonicated at temperature in a range from about 35-45° C., preferably about 40° C. for approximately one hour. Isophorone diisocyanate (IPDI) is mixed with water is added in dropwise to a beaker containing the water, surfactant (e.g., SDS), and hexadecane. The solution is sonicated to form the emulsion. 1,6-hexanediol (HDOH), or similar hydroxy-containing compound, and the at least one molecule to be encapsulated are dissolved in water and added to the solution with sonication. It is then stirred overnight at 40° C. to form the polyurethane. Light exposure is preferably minimized or eliminated. The nanocapsules can be collected by centrifugation and washed before flash freezing and lyophilization.

One of the important aspects of the polyurethane nanocapsules described herein is that they can be lyophilized, stored for long periods of time, and resuspended just prior to use. All of the formulations studied herein were lyophilized before either long-term or on-demand release studies were performed, although it should be appreciated by the person skilled in the art that the nanocapsules can be used immediately following synthesis, i.e., without being lyophilized. Being able to lyophilize, store, and resuspend nanocapsules increased their ability to be deployed and used in a number of environments and applications. Further, the polyurethane nanocapsules are biocompatible and biodegradable.

Advantageously, the ultrasound-triggered nanocapsules described herein can be administered intravenously [2, 53]. Polyurethane nanoparticles have been used intravenously in a number of applications and appear to clear without issue [54].

It should be appreciated by the person skilled in the art that the molecule encapsulated polyurethane nanocapsules described herein can be administered via other routes as well including, but not limited to, intravenous, intraarterial, intrathecal, intradermal, intracavitary, oral, rectal, intramuscular, subcutaneous, intracisternal, intravaginal, intraperitonial, intravitreal, suprachoroidal, subconjunctival, topical, buccal, and/or nasal routes of administration. The route of administration may also impact the dosage requirements.

### AMD and Treatment Thereof

AMD is the leading cause of blindness in the United States [55-56]. While most of the patients exhibit non-neovascular dry AMD, 10-15% of AMD patients display an exudative form of the disease (wet AMD) associated with choroidal (subretinal) neovascular angiogenesis. This choroidal neovascularization (CNV) is associated with subretinal fluid leakage, retinal scarring, and rapid vision loss [57]. Clinical therapies for wet AMD typically involve the intraocular delivery of antibodies or inhibitors to vascular endothelial growth factor (VEGF) as VEGF expression increases due to choroidal neovascularization in wet AMD [58]. Some of the anti-vascular endothelial growth factor (anti-VEGF) therapies that are currently used to treat AMD treatment include pegaptanib [59], ranibizumab [60-61], bevacizumab [60], and aflibercept [62], administered through intravitreal injections every four to six weeks. Disadvantageously, approximately 50% of patients receiving anti-VEGF therapy exhibit persistent disease activity (PDA), including edema, hemorrhage, and fibrosis, along with limited vision recovery. PDA leads to increased injections and increased risk of vision loss [76]. The quality of life of these patients will be deeply impacted by limiting daily activities like reading and driving [77]. Blocking VEGF only addresses the formation of leaky and new vessels in wet AMD. It does not address fibrosis [63, 78-80].

Pirfenidone is an FDA-approved small molecule drug that has been shown to reduce fibrosis in the eye [66],[69-70]. Reducing fibrosis in concert with blocking new vessels and leakiness may preserve vision and stop progression of wet AMD. Being able to do so using a long-term delivery system, that could be triggered in the physician's office to tailor delivery, may greatly improve AMD treatment as well as reduce the healthcare burden and increase patient adherence and outcomes [81-82]. Pirfenidone has been encapsulated in contact lenses using hydrogel chemistries for corneal burns [83, 84]. It has also been encapsulated in poly(lactic-co-glycolic acid) nanoparticles [73], chitosan/alginate particles [74], and liposomes [75]. These have been shown to deliver the drug for only a week or less.

Acriflavine has been shown to reduce CNV when given as a bolus administration intravitreally or suprachoroidally in rodents [38]. The challenge lies in the acriflavine half-life. Even by suprachoroidal administration, relief is on the order of days, and systemic delivery of the drug, as by topical administration, will have off-target effects. Acriflavine has been encapsulated in both polyester nanoparticles and microparticles. The particles delivered the drug for seven weeks and, following either intravitreal or suprachoroidal administration, led to a reduction in CNV as marked by fluorescein out to 9 weeks post-administration [85]. Disadvantageously, polyester particles are complement activators [86-87], which can exacerbate angiogenesis associated with AMD [88]. This potential for complement activation motivates the use of another platform that does not trigger complement.

Unlike the systems of the prior art, the polyurethane nanocapsules described herein can deliver a drug for several weeks following the administration of just one dose or bolus of the nanocapsules.

In a second aspect, the present invention relates to a method of inhibiting neovascularization in an eye of a subject in need thereof, said method comprising administering a long-term delivery system comprising acriflavine-encapsulated polyurethane nanocapsules to the eye of said subject. Alternatively, the method of the second aspect can relate to a method of treating wet-AMD, or preserving vision, in an eye of a subject in need thereof, said method comprising administering a long-term delivery system comprising acriflavine-encapsulated polyurethane nanocapsules to the eye of said subject. Preferably, the acriflavine-encapsulated polyurethane nanocapsules can be administered intravitreally, suprachoroidally, or subcunjunctivally, although other administration routes can be used. The administration of the acriflavine-encapsulated polyurethane nanocapsules can substantially slow or stop the progression of wet AMD, thus preserving vision. The long-term delivery system will reduce the number of injections to the eye. Without being bound by theory, it is assumed that the acriflavine substantially eliminates angiogenesis in the eye of a subject, relative to a control substantially devoid of acriflavine.

Advantageously, the acriflavine-encapsulated polyurethane nanocapsules can passively deliver the acriflavine over more than 4 weeks, more than 8 weeks, more than 12 weeks, or more than 16 weeks from the degradable polyurethane system, and/or they can be repeatedly triggered to release highly controlled and reproducible amounts of acriflavine in response to an amount of ultrasound energy applied to the nanocapsules. Accordingly, in one embodiment, the present invention relates to a method of inhibiting neovascularization in an eye of a subject in need thereof, said method comprising administering a long-term delivery system comprising acriflavine-encapsulated polyurethane nanocapsules to the eye of said subject; and applying ultrasound energy in proximity of the eye to release an amount of acriflavine therein. Alternatively, the method of the second aspect can relate to a method of treating wet-AMD, or preserving vision, in an eye of a subject in need thereof, said method comprising administering a long-term delivery system comprising acriflavine-encapsulated polyurethane nanocapsules to the eye of said subject; and applying ultrasound energy in proximity of the eye to release an amount of acriflavine therein. Preferably, the acriflavine-encapsulated polyurethane nanocapsules can be administered intravitreally, suprachoroidally, or subcunjunctivally, although other administration routes can be used. The administration of the acriflavine-encapsulated polyurethane nanocapsules can substantially slow or stop the progression of wet AMD, thus preserving vision. The long-term delivery system will reduce the number of injections to the eye. Without being bound by theory, it is assumed that the acriflavine substantially eliminates angiogenesis in the eye of a subject, relative to a control substantially devoid of acriflavine.

In a third aspect, the present invention relates to a method of substantially reducing or eliminating fibrosis in an eye of a subject in need thereof, said method comprising administering a long-term delivery system comprising pirfenidone-encapsulated polyurethane nanocapsules to the eye of said subject. Alternatively, the method of the third aspect can relate to a method of treating wet-AMD, or preserving vision, in an eye of a subject in need thereof, said method comprising administering a long-term delivery system comprising pirfenidone-encapsulated polyurethane nanocapsules to the eye of said subject. Preferably, the pirfenidone-encapsulated polyurethane nanocapsules can be administered intravitreally, suprachoroidally, or subcunjunctivally, although other administration routes can be used. The administration of the pirfenidone-encapsulated polyurethane nanocapsules can substantially slow or stop the progression of wet AMD, thus preserving vision. The long-term delivery system will reduce the number of injections to the eye. Without being bound by theory, it is assumed that the pirfenidone substantially eliminates scarring and/or reduces angiogenesis in the eye of a subject, relative to a control substantially devoid of pirfenidone.

Advantageously, the pirfenidone-encapsulated polyurethane nanocapsules can passively deliver the pirfenidone over more than 4 weeks, more than 8 weeks, more than 12 weeks, or more than 16 weeks from the degradable polyurethane system, and/or they can be repeatedly triggered to release highly controlled and reproducible amounts of pirfenidone in response to an amount of ultrasound energy applied to the nanocapsules. Accordingly, in one embodiment, the present invention relates to a method of substantially reducing or eliminating fibrosis in an eye of a subject in need thereof, said method comprising administering a long-term delivery system comprising pirfenidone-encapsulated polyurethane nanocapsules to the eye of said subject; and applying ultrasound energy in proximity of the eye to release an amount of acriflavine therein. Alternatively, the method of the third aspect can relate to a method of treating wet-AMD, or preserving vision, in an eye of a subject in need thereof, said method comprising administering a long-term delivery system comprising pirfenidone-encapsulated polyurethane nanocapsules to the eye of said subject; and applying ultrasound energy in proximity of the eye to release an amount of acriflavine therein. Preferably, the pirfenidone-encapsulated polyurethane nanocapsules can be administered intravitreally, suprachoroidally, or subcunjunctivally, although other administration routes can be used. The administration of the pirfenidone-encapsulated polyurethane nanocapsules can substantially slow or stop the progression of wet AMD, thus preserving vision. The long-term delivery system will reduce the number of injections to the eye. Without being bound by theory, it is assumed that the pirfenidone substantially eliminates scarring and/or reduces angiogenesis in the eye of a subject, relative to a control substantially devoid of pirfenidone.

In a fourth aspect, the present invention relates to a method of inhibiting neovascularization and substantially reducing or eliminating fibrosis in an eye of a subject in need thereof, said method comprising administering a combination therapy or dual delivery system comprising acriflavine-encapsulated polyurethane nanocapsules and pirfenidone-encapsulated polyurethane nanocapsules to the eye of said subject. Alternatively, the method of the fourth aspect can relate to a method of treating wet-AMD, or preserving vision, in an eye of a subject in need thereof, said method comprising administering a combination therapy or dual delivery system comprising acriflavine-encapsulated polyurethane nanocapsules and pirfenidone-encapsulated polyurethane nanocapsules to the eye of said subject. Preferably, both the acriflavine-encapsulated polyurethane nanocapsules and the pirfenidone-encapsulated polyurethane nanocapsules can be administered intravitreally, suprachoroidally, or subcunjunctivally. The administration of the combination therapy can substantially slow or stop the progression of wet AMD, thus preserving vision. It should be appreciated by the person skilled in the art that the acriflavine-encapsulated polyurethane nanocapsules and pirfenidone-encapsulated polyurethane nanocapsules can be administered in the eye of the subject simultaneously in a bolus injection or in separate injections. Further, it should be appreciated by the person skilled in the art that the acriflavine-encapsulated polyurethane nanocapsules can be distinct from the pirfenidone-encapsulated polyurethane nanocapsules or the polyurethane nanocapsules can comprise both acriflavine and pirfenidone encapsulated therein. Advantageously, the combination therapy or dual delivery system is a long-term delivery system that will reduce the number of injections to the eye. Without being bound by theory, it is assumed that the combination therapy comprising pirfenidone and acriflavine substantially eliminates scarring and/or reduces angiogenesis in the eye of a subject, relative to a control substantially devoid of pirfenidone and acriflavine.

Advantageously, the pirfenidone-encapsulated polyurethane nanocapsules and the acriflavine-encapsulated polyurethane nanocapsules can passively deliver the pirfenidone/acriflavine over more than 4 weeks, more than 8 weeks, more than 12 weeks, or more than 16 weeks from the degradable polyurethane system, and/or they can be repeatedly triggered to release highly controlled and reproducible amounts of pirfenidone/acriflavine in response to an amount of ultrasound energy applied to the nanocapsules. Accordingly, in another alternative, the present invention relates to a method of inhibiting neovascularization and substantially reducing or eliminating fibrosis in an eye of a subject in need thereof, said method comprising administering a combination therapy or dual delivery system comprising acriflavine-encapsulated polyurethane nanocapsules and pirfenidone-encapsulated polyurethane nanocapsules to the eye of said subject; and applying ultrasound energy in proximity of the eye to release an amount of acriflavine and an amount of pirfenidone therein. In yet another alternative, the method of the fourth aspect can relate to a method of treating wet-AMD, or preserving vision, in an eye of a subject in need thereof, said method comprising administering a combination therapy or dual delivery system comprising acriflavine-encapsulated polyurethane nanocapsules and pirfenidone-encapsulated polyurethane nanocapsules to the eye of said subject; and applying ultrasound energy in proximity of the eye to release an amount of acriflavine and an amount of pirfenidone therein. Preferably, both the acriflavine-encapsulated polyurethane nanocapsules and the pirfenidone-encapsulated polyurethane nanocapsules can be administered intravitreally, suprachoroidally, or subcunjunctivally. The administration of the combination therapy can substantially slow or stop the progression of wet AMD, thus preserving vision. It should be appreciated by the person skilled in the art that the acriflavine-encapsulated polyurethane nanocapsules and pirfenidone-encapsulated polyurethane nanocapsules can be administered in the eye of the subject simultaneously in a bolus injection or in separate injections. Further, it should be appreciated by the person skilled in the art that the acriflavine-encapsulated polyurethane nanocapsules can be distinct from the pirfenidone-encapsulated polyurethane nanocapsules or the polyurethane nanocapsules can comprise both acriflavine and pirfenidone encapsulated therein. Advantageously, the combination therapy or dual delivery system is a long-term delivery system that will reduce the number of injections to the eye. Without being bound by theory, it is assumed that the combination therapy comprising pirfenidone and acriflavine substantially eliminates scarring and/or reduces angiogenesis in the eye of a subject, relative to a control substantially devoid of pirfenidone and acriflavine.

The weight ratio of acriflavine to pirfenidone in the combination therapy or dual delivery system is in a range from about 1:10 to 10:1. More specifically, weight ratio of acriflavine to pirfenidone in the combination therapy or dual delivery system is in a range selected from about 1:10 to 1:9, about 1:9 to 1:8, about 1:8 to 1:7, about 1:7 to about 1:6, about 1:6 to about 1:5, about 1:5 to 1:4, about 1:4 to 1:3, about 1:3 to 1:2, about 1:2 to 1:1, about 1:1, about 1:1 to 2:1, about 2:1 to 3:1, about 3:1 to 4:1, about 4:1 to 5:1, about 5:1 to 6:1, about 6:1 to 7:1, about 7:1 to 8:1, about 8:1 to 9:1, about 9:1 to 10:1, about 1:2 to 2:1, about 1:3 to 3:1, or about 1:4 to 4:1, as readily determined by the person skilled in the art.

It should be appreciated that the administration of the acriflavine-encapsulated polyurethane nanocapsules to the eye in the combination therapy or dual delivery system may be the same technique as, or different from, the administration of the pirfenidone-encapsulated polyurethane nanocapsules to the eye. In one embodiment, both are administered intravitreally. In another embodiment, both are administered suprachoroidally. In still another embodiment, both are administered subcunjunctivally. In yet another embodiment, the acriflavine-encapsulated polyurethane nanocapsules are administered intravitreally and the pirfenidone-encapsulated polyurethane nanocapsules are administered suprachoroidally. In another embodiment, the acriflavine-encapsulated polyurethane nanocapsules are administered suprachoroidally and the pirfenidone-encapsulated polyurethane nanocapsules are administered intravitreally. In still another embodiment, the acriflavine-encapsulated polyurethane nanocapsules are administered intravitreally and the pirfenidone-encapsulated polyurethane nanocapsules are administered subcunjunctivally. In still another embodiment, the acriflavine-encapsulated polyurethane nanocapsules are administered subcunjunctivally and the pirfenidone-encapsulated polyurethane nanocapsules are administered intravitreally. In yet another embodiment, the acriflavine-encapsulated polyurethane nanocapsules are administered subcunjunctivally and the pirfenidone-encapsulated polyurethane nanocapsules are administered suprachoroidally. In yet another embodiment, the acriflavine-encapsulated polyurethane nanocapsules are administered suprachoroidally and the pirfenidone-encapsulated polyurethane nanocapsules are administered subcunjunctivally.

The loading of the encapsulated molecule to be administered is in a range from about 5 mg nanocapsules per mL of solution to about 50 mg of nanocapsules per mL of solution. Embodiments include, but are not limited to, about 5 mg/mL to about 25 mg/mL solution of nanocapsules, about 25 mg/mL to about 50 mg/mL solution of nanocapsules, and about 15 mg/mL to about 30 mg/mL solution of nanocapsules. The effective amount is dependent on the method of administration (e.g., intravitreally, suprachoroidally, or subcunjunctivally) as well as which drugs are included (e.g., acriflavine, pirfenidone, or both) and the patient themselves, as readily understood by the person skilled in the art.

Advantageously, ultrasound can be focused non-invasively and at a precise depth with sub-millimeter precision. Ultrasound can readily propagate to distances ranging from tens of cm in the MHz range to several meters in the kHz range. The disclosure contemplates that various ultrasound parameters are utilized in the practice of the methods disclosed herein. Thus, parameters including, but not limited to, frequency, pulse repetition frequency (e.g., from about 1 to about 50 Hz), and the number of cycles (e.g., from about 1 to about 100) per pulse are contemplated for use according to the methods described herein. As described above, the disclosure contemplates that ultrasound frequencies between about 0.25 MHz and about 50 MHz, or from about 0.25 MHz to about 10 MHz, are useful in the methods disclosed herein to enable efficient release of the molecules from the polyurethane nanocapsules. The disclosure contemplates that ultrasound pulse repetition frequencies (i.e., the number of ultrasound pulses per unit time) between about 1 Hertz (Hz) and about 50 Hz are useful in the methods disclosed herein. Further contemplated by the disclosure are embodiments in which the ultrasound pulse repetition frequency is at least 5 Hz, at least 10 Hz, at least 15 Hz, at least 20 Hz, at least 25 Hz, at least 30 Hz, at least 35 Hz, at least 40 Hz, or at least 45 Hz. Ultrasound pressure amplitudes less than 5 MPa are contemplated, e.g., 3 MPa or less, 2.5 MPa or less, or 2 MPa or less.

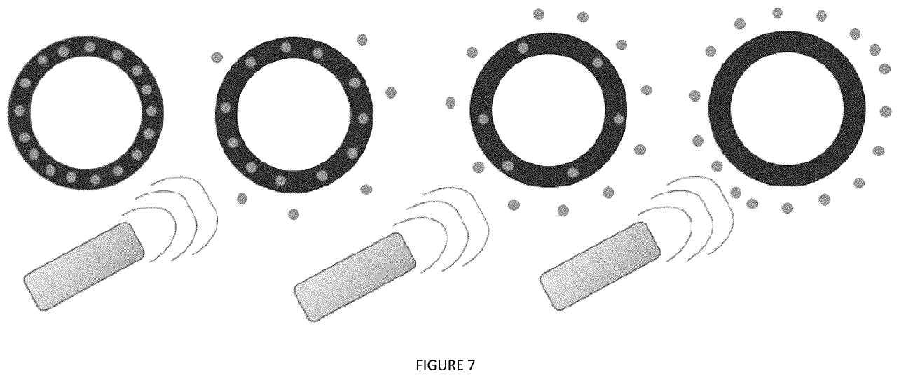

The on-demand release was experimentally determined using a sonicating probe. Advantageously, the settings used were consistent with high intensity focused ultrasound where the frequencies can be in the kilohertz range for tissue penetration and the power is often 100 W/cm² or higher [46]. In contrast, ultrasound for imaging is typically 2-3 MHz and limited to 0.72 W/cm² by the FDA [Id.]. A simplified schematic of the ultrasound process is shown in FIG. 7.

### Cancer and Treatment Thereof

Acriflavine and pirfenidone are known to have some potential anti-cancer applications. Towards that end, in a fifth aspect, the polyurethane nanocapsules encapsulating at least one molecule (e.g., a drug) can be used to prevent or treat cancer. For example, the acriflavine-encapsulated polyurethane nanocapsules can be used in the prevention or treatment of cancer including, but not limited to, brain cancer, pancreatic cancer, lung cancer, colorectal cancer, and melanoma. The acriflavine-encapsulated polyurethane nanocapsules can be the primary active ingredient in the treatment or can be combined with another known cancer-treating compound. For example, acriflavine-encapsulated polyurethane nanocapsules can be combined with 5-fluorouracil in the prevention or treatment of colorectal cancer.

Alternatively, the pirfenidone-encapsulated polyurethane nanocapsules can be used in the prevention or treatment of non-small cell lung cancer (NSCLC). The pirfenidone-encapsulated polyurethane nanocapsules can be the primary active ingredient in the treatment or can be combined with another known cancer-treating compound. For example, pirfenidone-encapsulated polyurethane nanocapsules can be combined with atezolizumab for the treatment of NSCLC.

In still another alterative, a combination therapy or dual delivery system comprising acriflavine-encapsulated polyurethane nanocapsules and pirfenidone-encapsulated polyurethane nanocapsules is used to prevent or treat cancer in a subject in need of said prevention or treatment. For example, the combination therapy or dual delivery system can be used to prevent or treat lung cancer.

Advantageously, the pirfenidone-encapsulated polyurethane nanocapsules and the acriflavine-encapsulated polyurethane nanocapsules can passively deliver the pirfenidone and/or acriflavine over more than 4 weeks, more than 8 weeks, more than 12 weeks, or more than 16 weeks from the degradable polyurethane system, and/or they can be repeatedly triggered to release highly controlled and reproducible amounts of pirfenidone and/or acriflavine in response to an amount of ultrasound energy applied to the nanocapsules. Accordingly, the method of the fifth aspect, regardless of the polyurethane nanocapsule used, can further comprise the application of ultrasound energy in proximity of the cancer cells or tumor to release an amount of the specific drug therein. It should be appreciated that “in proximity of the cancer cells or tumor” corresponds to within no more than 5 cm, preferably no more than 4 cm from the cancer cells or tumor, wherein the ultrasound probe is external to the body (e.g., the point of contact is directly on the skin) or inserted into a nearby cavity. In one embodiment, a coupling medium, as previously introduced, is applied between the ultrasound probe and the point of contact to maximize transmission.

### Conclusion

In conclusion, a system is described herein based on polyurethane nanocapsules as a platform for long-term, noninvasive, and on-demand delivery. Nanocapsules can be synthesized encapsulating a molecule such as a drug. The nanocapsules deliver their drugs passively over several weeks from a degradable polyurethane system, and/or they can be repeatedly triggered at least ten different times, over several days or weeks, to release highly controlled and reproducible amounts of drug in response to the amount of energy over time applied to the nanocapsules via ultrasound. Furthermore, the nanocapsules can be triggered to release on demand over a wide range of frequencies from the kilohertz range to the megahertz range suggesting that their on-demand behavior can be triggered using a variety of different ultrasound techniques. This makes the nanocapsules a highly versatile delivery system for both long-term, noninvasive, and on-demand delivery. A long-term formulation that delivers both an inhibitor of angiogenesis (e.g., acriflavine) and antifibrotic (e.g., pirfenidone) has the potential to preserve vision for longer times with fewer medical visits which may improve treatment outcomes but may also help to address the inequities in treatment.

The features and advantages of the invention are more fully illustrated by the following non-limiting examples, wherein all parts and percentages are by weight, unless otherwise expressly stated.

### Example 1

### Materials

All materials were obtained from commercial suppliers and used without further purification. Sodium dodecyl sulfate (SDS) (BP166, Fisher Scientific) and 99% pure hexadecane (AC120465000, ACROS Organics) were the surfactant and costabilizer, respectively, used to form the polyurethane nanocapsules. Isophorone diisocyanate (IPDI) 98% (AC427602500, ACROS Organics) and 1,6-hexanediol (HDOH) 97% (AAA1243930, ACROS Organics) were the reactants from which polyurethane was formed. Fluorescein (free acid, dye content 95%) (F2456, Sigma-Aldrich) was the fluorescent molecule that was encapsulated in the polyurethane shell and used to quantify the total mass that the polyurethane nanocapsules can encapsulate and release. Phosphate buffered solution (PBS) was used in the release study made via PBS tablets (P4417, Millipore Sigma). Both acriflavine and pirfenidone were obtained from Sigma-Aldrich.

### Characterization of Nanocapsules

a. Capsule Morphology and Size

A vacuum sputter coater (Denton Desk II) was used to deposit a 20 nm layer of gold palladium onto the nanocapsule samples placed on carbon tape on a specimen stub for scanning electron microscopy (SEM) imaging using the Nova NanoSEM 450 from FEI. The surface morphology of the capsules was examined as well as the diameters of the capsules. Samples for transmission electron microscopy (TEM) were prepared by adding lyophilized nanocapsules to a carbon-coated copper grid. TEM was performed using a FEI Morgagni M268 100 kV TEM equipped with a Gatan Orius CCD camera.

b. Capsule Size and Zeta Potential

The Malvern ZetaSizer (Nano ZS90) was used to determine the diameter and calculated zeta potential of the nanocapsules via dynamic light scattering (DLS). The nanocapsules were placed in a 1 mg/mL solution of 190 proof ethanol for sizing. This solution was pipetted into a cuvette (14955129, Fisher Scientific) which was placed into the ZetaSizer. The nanocapsules were placed in a 1 mg/mL solution of 10 mM potassium chloride (KCl) to determine the zeta potential. To measure the zeta potential, the solution was inserted into a folded capillary zeta cell (Malvern Store, DTS1070) which was placed in the ZetaSizer.

Both the size and zeta potential measurements were run in triplicates.

c. Capsule Molecular Components and Structures

Fourier-transform infrared (FT-IR) (PerkinElmer Frontier Optica) spectroscopy was used to produce spectra to identify the molecular components and structures associated with the capsules.

d. Gel Permeation Chromatography

Gel Permeation Chromatography (GPC) (Viscotek, V E 2001) was used to measure the molecular weight and size of the components of the capsules. The GPC column (PL1110-6504) is of the Agilent Plgel MIXED family. Its phase was MIXED-D, its inner diameter was 7.5 mm, its length was 300 mm, and its particle size was 5 m. To measure molecular weight and size, the reagents and capsule samples were first crushed via mortar and pestle. Then, a 10 mg of the crushed sample was resuspended in 1 mL of tetrahydrofuran (THF). This solution was filtered with PTFE membrane syringe filters (Fisher, 09-720-002) into GPC vials (VWR, 89239-024) which were placed in the machine for testing.

e. Characterization of Capsule Response to Sonication

10 mg of the fluorescein capsules were suspended in PBS and the solution was sonicated with a 130-Watt Ultrasonic Processor with Thumb-actuated Pulser at an amplitude of 76% for 30 seconds. After sonication, the solution was centrifuged again, the supernatant was removed and measured on a fluorescent plate reader (Molecular Devices, SpectraMax M2) (excitation max: 485; emission max 525). New PBS was added to resuspend the pellet. Then, the solution was inserted into a rotator in an oven at 37° C. for 15 minutes. After 15 minutes, the solution was again centrifuged, the supernatant was removed and tested, and new PBS was added to resuspend the pellet. The sonication and waiting were repeated until the fluorescent readings were unmeasureable. This entire process was repeated on another set of samples to determine the effect of the sonication time length. The only difference in this process was that the samples were sonicated at an amplitude of 76% for 1 minute. In between samples, the sonication probe was washed with acetone and dried off with a kimwipe or paper towel.

f. Sonication-Induced Release of Acriflavine Nanocapsules

10 mg of nanocapsules were placed in Eppendorf tubes with 1 ml of PBS. All experiments were performed in triplicate. The nanocapsules were exposed to 20 seconds of sonication at 50% amplitude. They were then centrifuged, the supernatant collected, and the pellet resuspended in fresh PBS. Samples were stored at −80° until they were read on the plate reader (excitation: 470 nm and emission: 513 nm). In between samples, the sonication probe was washed with acetone and dried off with a kimwipe or paper towel.

g. Characterization of Release of Drugs from Nanocapsules over Time

10 mg of the fluorescent capsules were suspended in PBS and the solution was placed in a rotator in an oven at 37° C. At specific time points, the solution was centrifuged for 10 minutes at 13.3 rpm, the supernatant was removed and stored in darkness at 4° C., and the pellet was resuspended in fresh PBS. The supernatant was then run on a fluorescent plate reader (Molecular Devices, SpectraMax M2) (excitation max: 490; emission max 525). This washing process was repeated until the optical density was below 20. At this point, the pellet was again resuspended in fresh PBS

Acriflavine release was performed in the same manner and the supernatant was measured on the plate reader excitation: 470 nm emission: 513 nm.

h. Ultrasound-Induced Release

The EYE CUBED ultrasound imaging system (Ellex; Mawson Lakes, Australia) was used with the ocular probe in the B scan mode at 90 dB. 10 mg of acriflavine nanocapsules were added to 20 ml of PBS in 50 ml conical tubes. Three replicates were tested at each timepoint (15 second of ultrasound, 30 seconds of ultrasound, and 60 seconds of ultrasound). The nanocapsules were held in PBS at room temperature for 90 minutes while getting access to the instrument. The probe was immersed in the solution for the designated time. Samples were then centrifuged, and the amount of acriflavine was determined using the SpectraMax M2 Microplate Reader (Molecular Devices LLC) in a 384-well Greiner black/clear plate. The excitation and emission wavelengths were 470 nm and 513 nm, respectively.

### Fluorescein-Encapsulated Polyurethane Nanocapsules

Fluorescein-encapsulated polyurethane nanocapsules were synthesized following the methodology of Torini et al. [23] and Guo et al. [24]. Creating polyurethane nanocapsules using an interfacial polymerization process has a long history for a range of applications, but it has never been used to create an ultrasound triggered on-demand delivery system. Polyurethane nanocapsules were formed via a poly-condensation in a two-phase system through mini-emulsions. Hexadecane and deionized water (DI) water formed the two phases, an oil phase and an aqueous phase. SDS was used as the surfactant to confer colloidal stability.

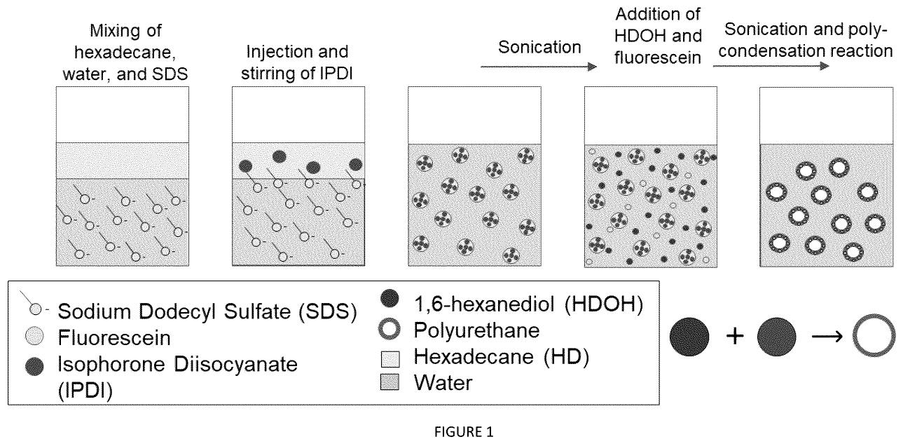

A schematic of the approach is shown in FIG. 1. Once 70 mL of water, 1.145 mL of hexadecane, and 1.1 g of surfactant (SDS) were mixed together at 300 rpm and 40° C. for 1 hour, 2.094 mL of IPDI was slowly dripped into the mixture and stirred; this step began the synthesis of the nanocapsules. By dripping the IPDI and monomers into the solution, the IPDI was evenly distributed throughout the oil phase. As the IPDI solution entered the pre-emulsification solution, the stirring speed was increased to 400 rpm. Once the IPDI solution was fully injected into the beaker, the solution was mixed at 400 rpm and 40° C. for 10 minutes. During this step, the solution remained clear. Next, the solution was sonicated with a 130-Watt Ultrasonic Processor with Thumb-actuated Pulser at an amplitude of 38% to break up any IPDI molecules that had aggregated. During this step, emulsions formed, and the solution looked like milk. While sonication was still progressing, an aqueous solution of 0.0013 g of fluorescein and 5.9 g of HDOH and 10 mL of DI water was dripped into the system. Because of the high reactivity of the isocyanate, the IPDI reacted immediately with the HDOH at the interface of the two phases.

After sonication, the solution was left to react for 24 hours at 40° C. and mixing at 300 rpm. After 24 hours, much of the solvent had evaporated and a clear, viscous solution was left in the beaker. The solution was poured into a centrifuge tube and centrifuged with DI water for a total of 5 runs at 10,000 rpm and 4° C. for 20 minutes each run. After each run, a distinct white pellet forms at the bottom of the tube. Additionally, after each run, the pale green supernatant was discarded, and new DI water was added to the tube, and the pellet was resuspended. After the fifth centrifugation period, the supernatant was again discarded, new DI water was added, and the pellet was then resuspended. The resuspended pellet was frozen in liquid nitrogen, wrapped in aluminum foil to protect the particles from light exposure, and lyophilized. When fully dry, the capsules appeared to be a white powder.

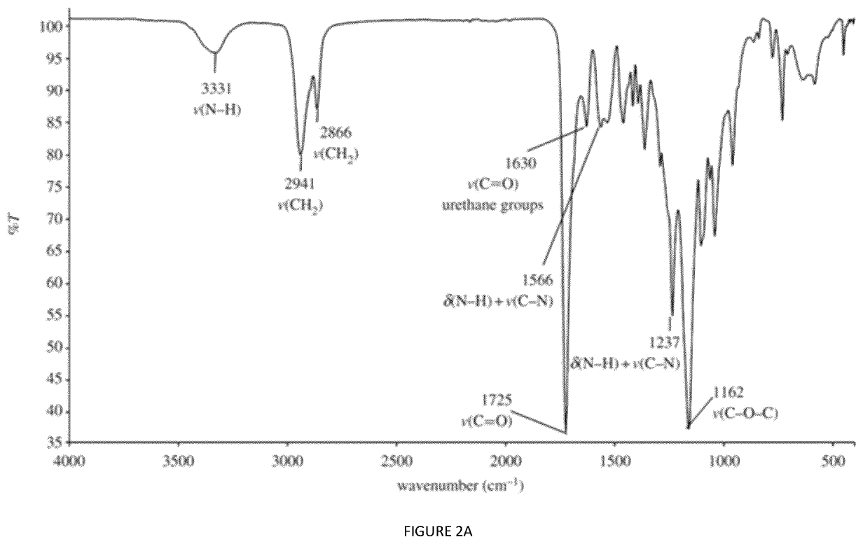

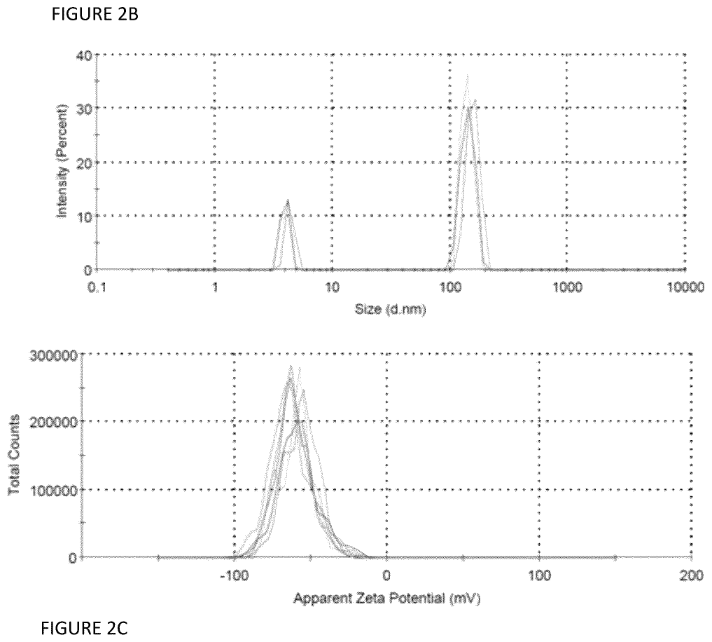

The FTIR spectrum (FIG. 2A) evidences the successful synthesis of fluorescein-encapsulated polyurethane nanocapsules. The peak at 1550 cm⁻¹ corresponds to the C—N vibration in the urethane and the peak at 1637 cm⁻¹ is due to the urea carbonyl presence in the nanocapsules. The peak at 1720 cm⁻¹ shows the C═O vibration, present in each capsule. Lastly, the peak at 3330 cm⁻¹ corresponds to the N—H vibration [25-27]. Similar spectra were obtained for both the fluorescein-encapsulated polyurethane nanocapsules and the acriflavine-encapsulated polyurethane nanocapsules. Dynamic Light Scattering (DLS) spectra in FIG. 2B show two peaks with one close to 4 nm, which is commonly seen in polyurethane syntheses [28-29], and the larger one, correlated with the mean effective diameter of the nanocapsules, at 145+/−9 nm. As seen in FIG. 2C, the zeta potential is extremely negative (−60 mV+/−12 mV), which is common with polyurethane-based materials made via emulsion polymerization because of the carboxyls at the surface as well as the SDS used to form the emulsions [30-32]. DLS was always performed after the particles had been lyophilized and then resuspended in ethanol or potassium chloride solution.

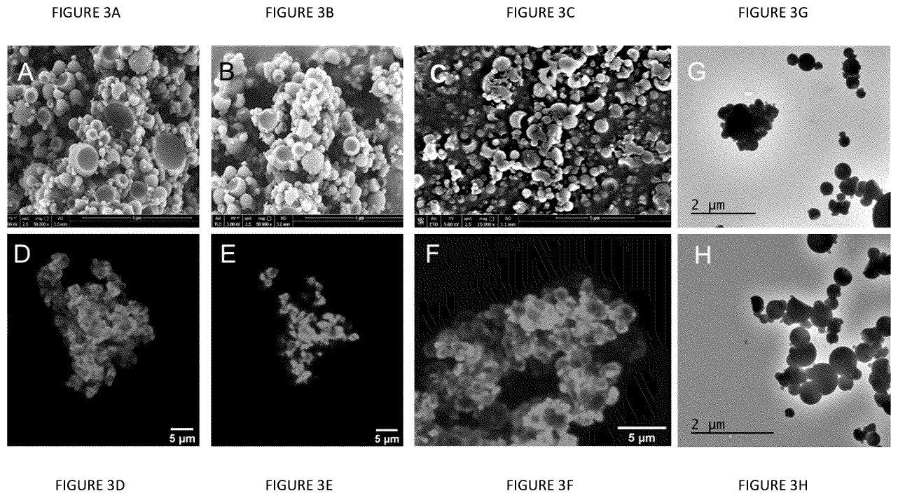

Scanning electron microscopy (SEM) was performed on the fluorescein-encapsulated polyurethane nanocapsules before (FIGS. 3A and 3B) and after sonication studies. The SEM results correlated with the DLS findings with the fluorescein-encapsulated polyurethane nanocapsules being around 150 nm in diameter with a notable distribution in sizes. Many of the capsules looked partially deflated. We saw this is all batches and attribute it to the lyophilization of the nanocapsules (see, e.g., FIGS. 3A and 3B). After sonication, we found that while there were some burst capsules, the majority remained intact. A repeated 60 second sonication burst study was performed wherein the nanocapsules exposed to 1 minute sonication at 20 W with 10 repetitions. The post-sonication image in shown in FIG. 3C, wherein the majority of the nanocapsules are intact, but there are some fragments that can be seen in the image.

Confocal microscopy demonstrated that the fluorescein was localized in the shells of the polyurethane nanocapsules (see, FIGS. 3D-3F). This is not surprising since fluorescein is a water-soluble molecule, and during the emulsification procedure, water-soluble molecules are likely to associate with the interfacial region of the nanoemulsion. For the confocal work, it was very difficult to resolve individual particles so the primary focus was to assess the localization of the encapsulated molecule or drug.

TEM of the nanocapsules showed a range of particle sizes consistent with the DLS data as well as some aggregates of nanocapsules (see, FIG. 3G) and individual nanocapsules (see, FIGS. 3G and 3H). The dry polyurethane nanocapsules often appeared aggregated but when resuspended, the particles dissociated.

Fluorescein Release and On-Demand Delivery from Fluorescein-Encapsulated Polyurethane Nanocapsules

The loading of fluorescein was approximately 0.012 ug of fluorescein per mg of nanocapsules, which based on the amounts initially added during synthesis, was approximately 10% of the amount added. The amount was more than enough to investigate the passive release of fluorescein at 37° C. from the fluorescein-encapsulated polyurethane nanocapsules versus the impact of sonication on release.

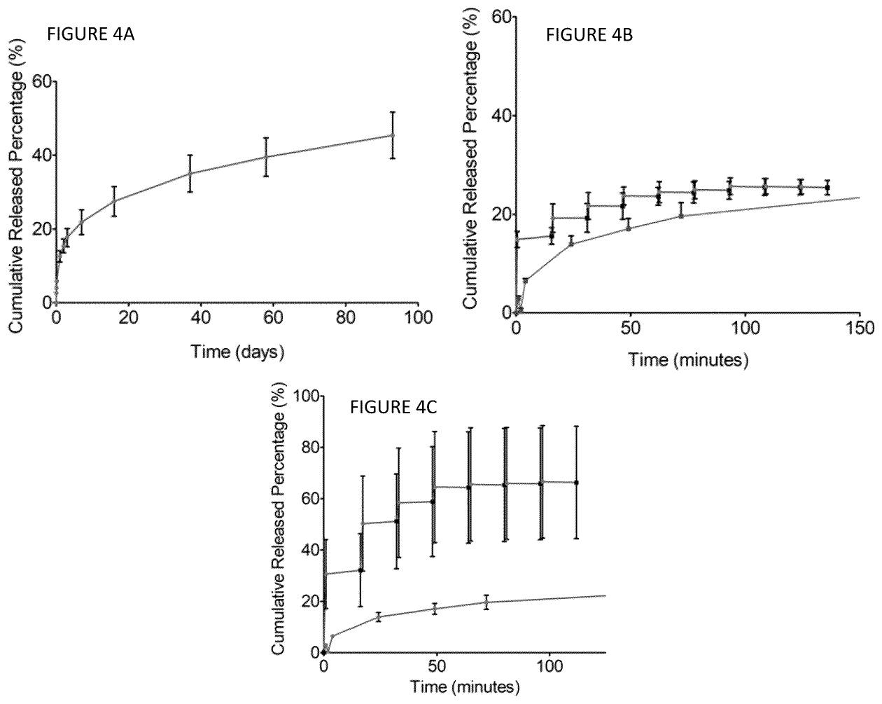

Normal, passive delivery from the fluorescein-encapsulated polyurethane nanocapsules led to release of the fluorescein for well over 90 days (see, FIG. 4A). At the end of 90 days, no capsules could be pelleted, and the release study was terminated. In all of our release studies carried to completion, the nanocapsules were degraded to the point where no more material could be collected. The synthesis of hexanediol with IPDI leads to urethane linkages, as confirmed by the FTIR in FIG. 1. Urethane linkages degrade either enzymatically [33-34] or by hydrolysis which is a significant limitation to using polyurethanes for long term applications in vivo, but it is a tremendous asset for a nanocapsule delivery system [35-37].

On-demand delivery was achieve by exposing the nanocapsules to a sonicating probe device at 70% amplitude and 20 kHz which correlates with 20 Watts or 70 W/cm² using a 6 mm probe. 30 second sonication events led to approximately 3-5% of the total amount of fluorescein being released (see, FIG. 4B). In contrast, 60 second sonication events led to ˜25% of the fluorescein being released (see, FIG. 4C). These findings suggest that one can tune the amount of a drug released based on the amount of sonication applied over time. Having not only an on-demand system but an addressable on demand system could be extremely helpful in the treatment of a number of conditions.

Although not wishing to be bound by theory, it is thought that the ultrasound triggers release of the encapsulated molecule from the polyurethane nanocapsules either through hyperthermia and/or through cavitation [52]. Early data over a wide range of energies and frequencies seems more consistent with cavitation than hyperthermia, but this remains to be determined conclusively in the future.

### Example 2

### Acriflavine Long-Term Release and On-Demand Delivery

Based on the findings hereinabove for fluorescein, the same encapsulation synthesis and sonication approach was applied to a clinically relevant drug, acriflavine (IUPAC: 3,6-diamino-10-methylacridin-10-ium chloride). Acriflavine was originally used as an antiseptic in WWII but has more recently been shown to be an effective inhibitor of HIF-1-alpha dimerization and angiogenesis [38-39]. Acriflavine has been encapsulated previously in a lipid nanocapsule formulation for targeting tumors with the majority being released in the first four hours [40].

Acriflavine was also encapsulated in the polyurethane nanocapsules using the synthesis shown in FIG. 1. A 250 mL propylene beaker containing 1.1 g of SDS dissolved in 70 mL of DI water was prepared. This SDS and DI water solution was mixed with 1.145 mL of hexadecane, and the solution was allowed to stir at 40° C. and 300 rpm for one hour. After an hour had passed, the stirring speed was raised to 400 rpm (with the temperature of 40° C. being maintained) and 2.094 mL of IPDI mixed with 330 mg of acriflavine dissolved in 3-7 mL of DI water was added in dropwise to the beaker containing the DI water, SDS, and hexadecane. This dropwise addition was achieved by using a glass syringe attached to a 20 G needle. After the dropwise addition of the entirety of the acriflavine/IPDI/DI water solution under gravity was complete, the solution was allowed to stir for an additional 10 minutes at 400 rpm. The beaker and its contents were then transferred to a hood containing a sonicator. The solution was sonicated for one minute at 38% amplitude. After one minute, the solution continued to be sonicated at 38% amplitude while HDOH dissolved in 10 mL of DI water was added into the beaker over the course of one minute for a total of two minutes of cumulative sonication. Following sonication, the beaker and its contents were transferred back to a hot plate, and the reaction was allowed to occur at 40° C. with a stirring speed of 300 rpm for 24 hours; this could be said to be a maturation step in which the IPDI and HDOH are allowed to react and form layers of polyurethane. During this step, the beaker was covered in aluminum foil to minimize light exposure.

Following the 24-hour maturation step, the formation of polyurethane had occurred. The polyurethane was found along the sides of the beaker as well as on the bottom of the beaker. What appears to be excess/unencapsulated acriflavine made some of the polyurethane appear a dark orange, while other polyurethane particles appeared light orange. All of the solid particles of polyurethane were transferred to a centrifuge tube and spun down for 10 minutes at 10,062× G. After centrifugation, a light orange pellet formed on the bottom and along the sides of the tube. Overtop of the pellet and the supernatant, a thin layer of hexadecane had formed, which was removed as completely as possible. Following removal of the hexadecane and the supernatant, enough fresh DI water was added to the tubes to cover the pellets. The DI water and pellet were then vortexed briefly (for roughly ten seconds) and the solution was once again spun down at 10,062×G. The same process of removing the supernatant and hexadecane and adding new DI water was repeated, and the solution was spun down for a third and final time. After the third round of centrifugation, the hexadecane was gone entirely, or present in small enough quantities to no longer be readily visible. The supernatant was removed once again, and the pellet of polyurethane was re-suspended in DI water. The suspension of polyurethane particles was then transferred to a pre-weighed 50 mL conical tube and was freeze-dried using liquid nitrogen. Following freeze-drying, the sample was wrapped in aluminum foil and lyophilized.

The acriflavine-encapsulated polyurethane nanocapsules were determined to have a mean effective diameter of 260+/−37 nm by DLS. The loading of the acriflavine-encapsulated polyurethane nanocapsules was approximately 54 ug of acriflavine per mg of nanocapsules. This is well within the therapeutic window for delivery of acriflavine to inhibit angiogenesis [40-42].

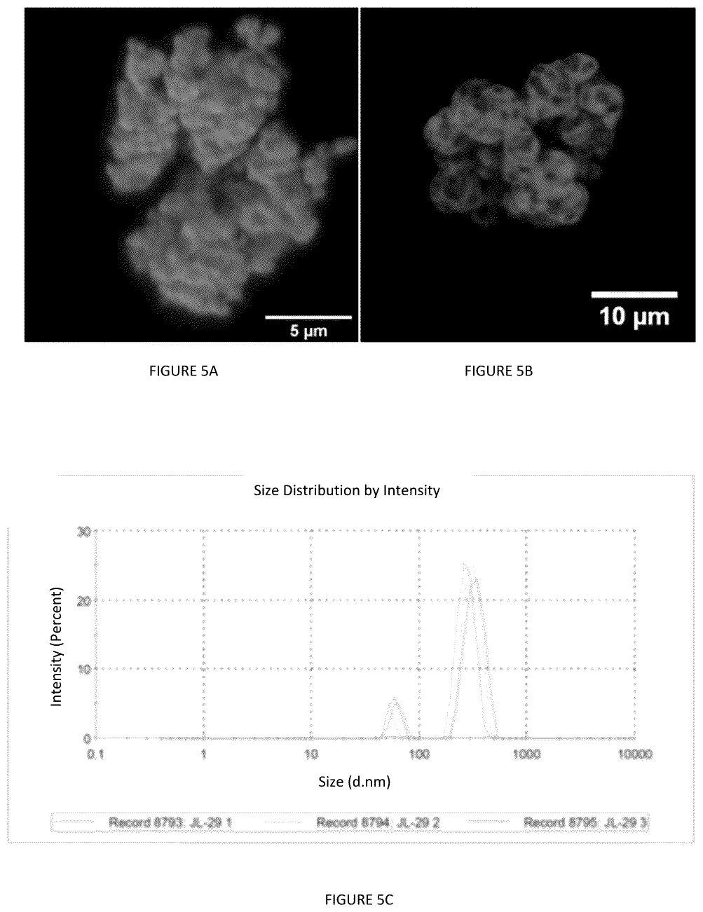

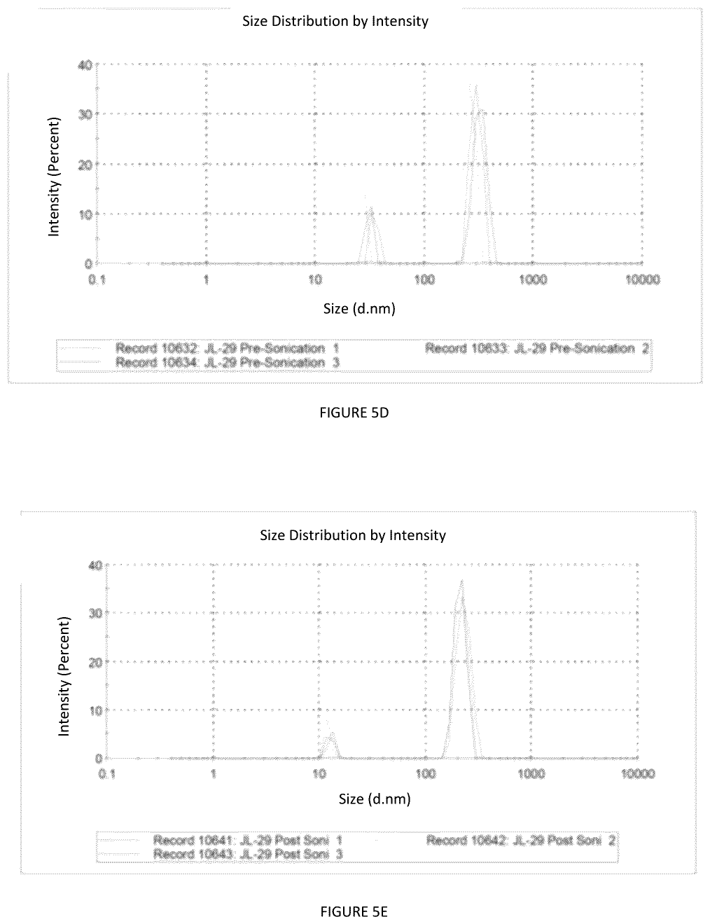

Acriflavine fluoresces with excitation at 416 nm and emission at 514 nm. Confocal microscopy was performed and it can be seen that the drug was localized in the shells of the nanocapsules, analogous to that seen with fluorescein. As with the fluorescein nanocapsules, clusters of acriflavine-encapsulated polyurethane nanocapsules were visualized in the confocal microscope (see, FIGS. 5A and B).

However, using DLS the nanocapsule size was found to be 295+/−33 nm with no signs of aggregates or large peaks even post lyophilization (FIG. 5C). Following 10 rounds of sonication, the nanocapsules had statistically similar dimensions according to DLS (FIG. 5E). This suggests that the acriflavine-encapsulated polyurethane nanocapsules are not aggregated in solution even after lyophilization and storage. It also suggests that they are remain intact, as was seen with the fluorescein nanocapsules even after repeated sonications.

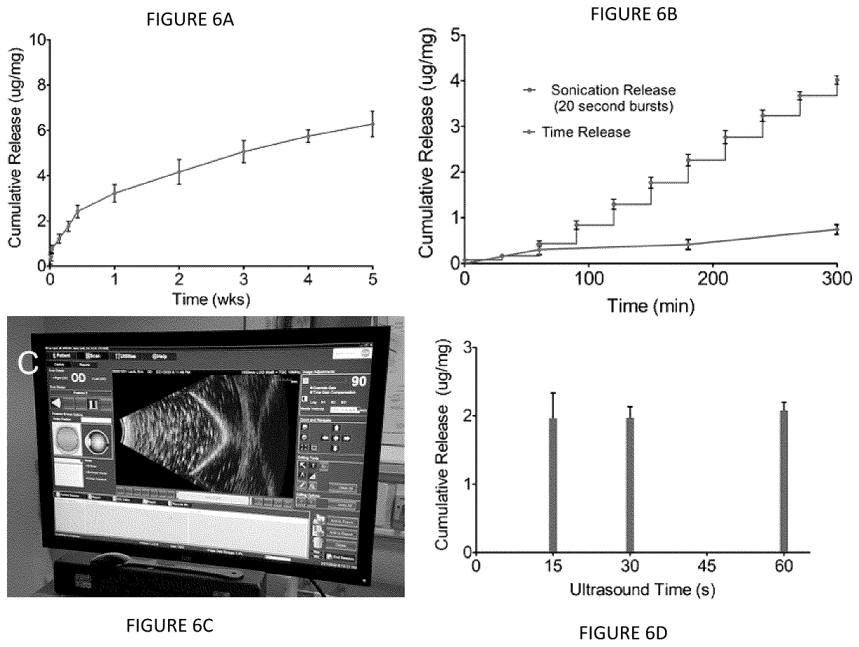

A standard infinite sink release from the acriflavine-encapsulated polyurethane nanocapsules was performed with approximately 10% acriflavine being released over the first 5 weeks (see, FIG. 6A). The release study was terminated after the fifth week due to the COVID-19 pandemic and the temporary closing of the research lab. Acriflavine is more hydrophobic than fluorescein which may account for the higher loading and slower release over time.

The acriflavine-encapsulated polyurethane nanocapsules were then sonicated using 20 second pulses at 30 minute intervals. The pulses consisted of 20 seconds of exposure to sonication at 50% amplitude, with a frequency of 20 kHz. The correlates with sonication at 10 Watts or 35 W/cm² based on probe dimensions. It was discovered that this led to a very tightly controlled, reproducible release of acriflavine at each sonication point with approximately 0.5 ug of acriflavine being released per mg of nanocapsules at each sonication point (see, FIG. 6B). These sonication pulses were overlaid with the first part of the long-term release curve of FIG. 6A to show the relative amount released over time in the absence and presence of sonication. It is clearly seen that sonication leads to pulses of release of acriflavine from the acriflavine-encapsulated polyurethane nanocapsules.

Being able to control the amount of drug released and being able to do so over 10 sonication steps over many hours is promising, but the question arose as to whether release could be controlled via a clinical instrument. The EYE CUBED instrument (Ellex Corporation, Australia). was used in the posterior B scan probe with the standard setting at 90 dB and 10 MHz. This setting is approved by the FDA for imaging the retina. The probe was placed into the solution with the acriflavine-encapsulated polyurethane nanocapsules and activated for 15, 30 or 60 seconds. Not surprisingly, the nanocapsules are bright on the ultrasound image with what appears to be high echogenicity much like their gas-containing microbubble counterparts [43-45]. Regardless of exposure time, the nanocapsules exhibited statistically similar release of approximately 2 ug of acriflavine per mg of nanocapsules. Not only can the nanocapsules be seen by ultrasound, but it is possible to trigger on-demand release even during an imaging setup. The FDA limits the energy produced by clinical imaging probes to 0.72 W/cm² [46], so even a low energy system can trigger on-demand release.

Surprisingly, the encapsulated molecules could be released, albeit in vitro, with an FDA-approved ocular diagnostic imaging system. The B scan mode used was 10 MHz, which is optimized for viewing the retina. Higher frequencies have less penetration but greater resolution than lower frequencies, so ocular imaging systems use the high frequency ultrasound [50-51]. It is important to note that the low energy of a clinical imaging system suggests that the nanocapsules described herein may be able to be used for drug delivery in spaces, such as the lung, where lower energies are needed for ultrasound to be safe and effective.

### Example 3

Approximately one-third of patients diagnosed with AMD-related choroidal neovascularization (CNV) exhibit fibrosis attributed to their continued vision loss despite anti-VEGF therapy [63]. The realization that fibrosis is a significant issue has sparked an interest in reducing or eliminating fibrosis in AMD as a complementary therapy to anti-VEGF therapies [64]. Likewise, there is a strong interest in modulating inflammation in AMD [Id.]. Pirfenidone (IUPAC name: 5-methyl-1-phenylpyridin-2-one) is an FDA-approved antifibrotic, anti-inflammatory drug with a strong safety profile [65]. It has been shown to reduce fibrosis when injected intravitreally 14 days after laser-induced CNV as well [66]. Pirfenidone down-regulates several inflammatory cytokines, including TGF-beta, IL-6 as well as bFGF [67]. Furthermore, pirfenidone has been shown to downregulate VEGF in vitro [68] and in vivo in the eye [69-71]. Pirfenidone has been shown to reduce angiogenesis in a model of CNV [70].

However, when administered to the eye topically, pirfenidone washes out very quickly, within minutes, with no measurable amounts in the back of the eye [72]. This washout has motivated the development of long-term delivery systems. So far, the majority of systems deliver the drug for one week or less [73-75]. In one aspect, the present inventors have encapsulated pirfenidone in polyurethane nanocapsules to deliver physiologically relevant amounts of pirfenidone for at least one months, preferably at least two months, and more preferably at least three months. In one embodiment, the pirfenidone-encapsulated polyurethane nanocapsules are to be administered intravitreally because it has been promising in a number of therapies for AMD.

Pirfenidone was encapsulated in the polyurethane nanocapsules using the synthesis shown in FIG. 1. Sodium dodecyl sulfate (SDS) was dissolved in water and hexadecane and stirred at 40° C. for one hour. Isophorone diisocyanate (IPDI) was mixed with pirfenidone and water was added in dropwise to the beaker containing the DI water, SDS, and hexadecane. The solution was sonicated to form the emulsion. HDOH is dissolved in water and added to the solution with sonication. It was then stirred overnight at 40° C. to form the polyurethane. The nanocapsules were collected by centrifugation and washed three times before flash freezing and lyophilization. Control nanocapsules were synthesized in the same manner except that pirfenidone was not included.

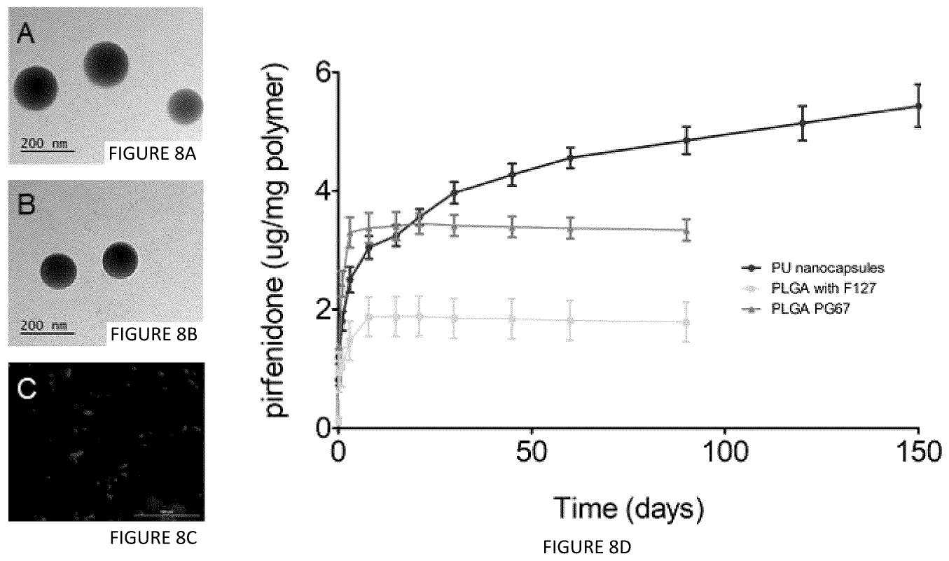

Nanocapsule size and distribution is determined using DLS. The mean effective diameter of the pirfenidone-encapsulated polyurethane nanocapsules was determined to be 245±40 nm. Zeta potential was measured in a KCl solution and was determined to be −50.4±12 mV. Control nanocapsules had a similar size and zeta potential. Particle shape was confirmed via SEM and TEM for both. Transmission electron microscopy demonstrated that the nanocapsules were spherical as expected (see, FIGS. 8A-8B.

Pirfenidone can be excited at 310 nm with emission at 410 nm. Therefore, nanocapsules with the drug can be imaged in the DAPI channel, and drug loading and release can be measured via a fluorimeter (see, FIG. 8C).

For the loading studies, 5 mg of pirfenidone-encapsulated polyurethane nanocapsules were dissolved in 1 ml DMSO. The fluorescence was determined using SpectraMax M2 Microplate Reader (Molecular Devices LLC, San Jose CA) for pirfenidone excitation and emission wavelengths in DMSO (excitation wavelength, λₑₓ=310 nm; emission wavelength, λₑₘ=410 nm). Loading was determined from a linear fitted calibration curve in the range of 0.0024-5 μg mL-1 of pirfenidone in DMSO. The loading of pirfenidone in the polyurethane nanocapsules was 31.5 ug/mg polymer. For the release study, 10 mg of the pirfenidone-encapsulated polyurethane nanocapsules were suspended in PBS, and the solution was placed in a rotator in an oven at 37° C. At specific time points, the solution was centrifuged, the supernatant was removed and stored in darkness at −20° C., and the pellet was resuspended in fresh PBS. The amount of pirfenidone is quantified in the supernatant using a fluorescent plate reader (Molecular Devices, SpectraMax M2) (excitation: 310 nm emission: 410 nm). Release curves using an infinite sink release system in FIG. 8D show that while PLGA-based nanoparticles deliver the drug for 7 days, the pirfenidone-encapsulated polyurethane nanocapsules release the drug for at least 150 days with 20% of the drug released.

Although the invention has been variously disclosed herein with reference to illustrative embodiments and features, it will be appreciated that the embodiments and features described hereinabove are not intended to limit the invention, and that other variations, modifications and other embodiments will suggest themselves to those of ordinary skill in the art, based on the disclosure herein. The invention therefore is to be broadly construed, as encompassing all such variations, modifications and alternative embodiments within the spirit and scope of the claims hereafter set forth.

### REFERENCES

1. Luo, Z.; Jin, K.; Pang, Q.; Shen, S.; Yan, Z.; Jiang, T.; Zhu, X.; Yu, L.; Pang, Z.; Jiang, X., On-Demand Drug Release from Dual-Targeting Small Nanoparticles Triggered by High-Intensity Focused Ultrasound Enhanced Glioblastoma-Targeting Therapy. ACS applied materials & interfaces 2017, 9 (37), 31612-31625.
2. Cullion, K.; Rwei, A. Y.; Kohane, D. S., Ultrasound-triggered liposomes for on-demand local anesthesia. Ther Deliv 2018, 9 (1), 5-8.
3. Tamayol, A.; Hassani Najafabadi, A.; Mostafalu, P.; Yetisen, A. K.; Commotto, M.; Aldhahri, M.; Abdel-Wahab, M. S.; Najafabadi, Z. I.; Latifi, S.; Akbari, M.; Annabi, N.; Yun, S. H.; Memic, A.; Dokmeci, M. R.; Khademhosseini, A., Biodegradable elastic nanofibrous platforms with integrated flexible heaters for on-demand drug delivery. Scientific reports 2017, 7 (1), 9220.
4. Li, J.; Sun, C.; Tao, W.; Cao, Z.; Qian, H.; Yang, X.; Wang, J., Photoinduced PEG deshielding from ROS-sensitive linkage-bridged block copolymer-based nanocarriers for on-demand drug delivery. Biomaterials 2018, 170, 147-155.
5. Lee, S.; Hwang, G.; Kim, T. H.; Kwon, S. J.; Kim, J. U.; Koh, K.; Park, B.; Hong, H.; Yu, K. J.; Chae, H.; Jung, Y.; Lee, J.; Kim, T. I., On-Demand Drug Release from Gold Nanoturf for a Thermo- and Chemotherapeutic Esophageal Stent. ACS Nano 2018, 12 (7), 6756-6766.
6. Yuan, Z.; Demith, A.; Stoffel, R.; Zhang, Z.; Park, Y. C., Light-activated doxorubicin-encapsulated perfluorocarbon nanodroplets for on-demand drug delivery in an in vitro angiogenesis model: Comparison between perfluoropentane and perfluorohexane. Colloids Surf B Biointerfaces 2019, 184, 110484.
7. Lee, S. H.; Kim, B. H.; Park, C. G.; Lee, C.; Lim, B. Y.; Choy, Y. B., Implantable small device enabled with magnetic actuation for on-demand and pulsatile drug delivery. J Control Release 2018, 286, 224-230.
8. Brudno, Y.; Mooney, D. J., On-demand drug delivery from local depots. J Control Release 2015, 219, 8-17.
9. Khorshidi, S.; Karkhaneh, A., On-demand release of ciprofloxacin from a smart nanofiber depot with acoustic stimulus. Journal of biosciences 2018, 43 (5), 959-967.
10. Ogawa, K.; Fuchigami, Y.; Hagimori, M.; Fumoto, S.; Maruyama, K.; Kawakami, S., Ultrasound-responsive nanobubble-mediated gene transfection in the cerebroventricular region by intracerebroventricular administration in mice. European journal of pharmaceutics and biopharmaceutics: official journal of Arbeitsgemeinschaft fur Pharmazeutische Verfahrenstechnik e.V 2019, 137, 1-8.
11. Jamburidze, A.; Huerre, A.; Baresch, D.; Poulichet, V.; De Corato, M.; Garbin, V., Nanoparticle-Coated Microbubbles for Combined Ultrasound Imaging and Drug Delivery. Langmuir: the ACSjournal of surfaces and colloids 2019, 35 (31), 10087-10096.
12. Ma, J.; Xu, C. S.; Gao, F.; Chen, M.; Li, F.; Du, L. F., Diagnostic and therapeutic research on ultrasound microbubble/nanobubble contrast agents (Review). Molecular medicine reports 2015, 12 (3), 4022-4028.
13. Wu, M.; Zhao, H.; Guo, L.; Wang, Y.; Song, J.; Zhao, X.; Li, C.; Hao, L.; Wang, D.; Tang, J., Ultrasound-mediated nanobubble destruction (UMND) facilitates the delivery of A10-3.2 aptamer targeted and siRNA-loaded cationic nanobubbles for therapy of prostate cancer. Drug delivery 2018, 25 (1), 226-240.
14. Huang, D.; Chen, Y.-S.; Thakur, S. S.; Rupenthal, I. D., Ultrasound-mediated nanoparticle delivery across ex vivo bovine retina after intravitreal injection. European journal of pharmaceutics and biopharmaceutics: official journal of Arbeitsgemeinschaft fur Pharmazeutische Verfahrenstechnik e.V 2017, 119, 125-136.
15. Song, Z.; Wang, Z.; Shen, J.; Xu, S.; Hu, Z., Nerve growth factor delivery by ultrasound-mediated nanobubble destruction as a treatment for acute spinal cord injury in rats. International journal of nanomedicine 2017, 12, 1717-1729.
16. Simões, M. G.; Hugo, A.; Alves, P.; Pérez, P. F.; Gómez-Zavaglia, A.; Simões, P. N., Long term stability and interaction with epithelial cells of freeze-dried pH-responsive liposomes functionalized with cholesterol-poly(acrylic acid). Colloids and surfaces. B, Biointerfaces 2018, 164, 50-57.
17. Payton, N. M.; Wempe, M. F.; Xu, Y.; Anchordoquy, T. J., Long-term storage of lyophilized liposomal formulations. Journal of pharmaceutical sciences 2014, 103 (12), 3869-3878.
18. Hernandez, C.; Nieves, L.; de Leon, A. C.; Advincula, R.; Exner, A. A., Role of Surface Tension in Gas Nanobubble Stability Under Ultrasound. ACS applied materials & interfaces 2018, 10 (12), 9949-9956.
19. Husseini, G. A.; Pitt, W. G.; Martins, A. M., Ultrasonically triggered drug delivery: breaking the barrier. Colloids and surfaces. B, Biointerfaces 2014, 123, 364-386.
20. Baghirov, H.; Snipstad, S.; Sulheim, E.; Berg, S.; Hansen, R.; Thorsen, F.; Mørch, Y.; Davies, C. d. L.; Åslund, A. K. O., Ultrasound-mediated delivery and distribution of polymeric nanoparticles in the normal brain parenchyma of a metastatic brain tumour model. PloS one 2018, 13 (1), e0191102-e0191102.
21. Ye, D.; Zhang, X.; Yue, Y.; Raliya, R.; Biswas, P.; Taylor, S.; Tai, Y.-C.; Rubin, J. B.; Liu, Y.; Chen, H., Focused ultrasound combined with microbubble-mediated intranasal delivery of gold nanoclusters to the brain. Journal of controlled release: official journal of the Controlled Release Society 2018, 286, 145-153.
22. Papa, A.-L.; Korin, N.; Kanapathipillai, M.; Mammoto, A.; Mammoto, T.; Jiang, A.; Mannix, R.; Uzun, O.; Johnson, C.; Bhatta, D.; Cuneo, G.; Ingber, D. E., Ultrasound-sensitive nanoparticle aggregates for targeted drug delivery. Biomaterials 2017, 139, 187-194.
23. Torini, L.; Argillier, J. F.; Zydowicz, N., Interfacial Polycondensation Encapsulation in Miniemulsion. Macromolecules 2005, 38 (8), 3225-3236.
24. GUO Jinxin, PAN Qiuhua, HUANG Cui, ZHAO Yanbing, OUYANG Xiaobai, HUO Yonghong, and DUAN Sansan, The Role of Surfactant and Costabilizer in Controlling Size of Nanocapsules Containing TEGDMA in Miniemulsion. Journal of Wuhan University of Technology—Mater. Sci. Ed. 2009, 24(6), 1004.
25. Hung, W. C.; Shau, M. D.; Kao, H. C.; Shih, M. F.; Cherng, J. Y., The synthesis of cationic polyurethanes to study the effect of amines and structures on their DNA transfection potential. Journal of Controlled Release 2009, 133 (1), 68-76.
26. Sobczak, M., Synthesis and Characterization of Polyurethanes Based on Oligo(ϵ-caprolactone) Prepared by Free-Metal Method. Journal of Macromolecular Science, PartA 2011, 48 (5), 373-380.
27. Ouyang, X.; Huang, X.; Pan, Q.; Zuo, C.; Huang, C.; Yang, X.; Zhao, Y., Synthesis and characterization of triethylene glycol dimethacrylate nanocapsules used in a self-healing bonding resin. Journal of dentistry 2011, 39 (12), 825-33.
28. Su, J. F.; Wang, L. X.; Ren, L.; Huang, Z.; Meng, X. W., Preparation and characterization of polyurethane microcapsules containing n-octadecane with styrene-maleic anhydride as a surfactant by interfacial polycondensation. Journal of Applied Polymer Science 2006, 102 (5), 4996-5006.
29. Wu, J.; Weir, M. D.; Zhang, Q.; Zhou, C.; Melo, M. A. S.; Xu, H. H. K., Novel self-healing dental resin with microcapsules of polymerizable triethylene glycol dimethacrylate and N,N-dihydroxyethyl-p-toluidine. Dental materials: official publication of the Academy of Dental Materials 2016, 32 (2), 294-304.
30. Zhu, Q.; Wang, Y.; Zhou, M.; Mao, C.; Huang, X.; Bao, J.; Shen, J., Preparation of anionic polyurethane nanoparticles and blood compatible behaviors. J Nanosci Nanotechnol 2012, 12 (5), 4051-6.
31. Pabari, R. M.; Mattu, C.; Partheeban, S.; Almarhoon, A.; Boffito, M.; Ciardelli, G.; Ramtoola, Z., Novel polyurethane-based nanoparticles of infliximab to reduce inflammation in an in-vitro intestinal epithelial barrier model. Int J Pharm 2019, 565, 533-542.
32. Huang, Y. J.; Hung, K. C.; Hsieh, F. Y.; Hsu, S. H., Carboxyl-functionalized polyurethane nanoparticles with immunosuppressive properties as a new type of anti-inflammatory platform. Nanoscale 2015, 7 (48), 20352-64.
33. Tang, Y. W.; Labow, R. S.; Santerre, J. P., Enzyme-induced biodegradation of polycarbonate-polyurethanes: Dependence on hard-segment chemistry. Journal of Biomedical Materials Research 2001, 57 (4), 597-611.
34. Kreitz, M. R.; Domm, J. A.; Mathiowitz, E., Controlled delivery of therapeutics from microporous membranes. II. In vitro degradation and release of heparin-loaded poly(D,L-lactide-co-glycolide). Biomaterials 1997, 18 (24), 1645-51.
35. Żóltowska, K.; Piotrowska, U.; Oledzka, E.; Kuras, M.; Zgadzaj, A.; Sobczak, M., Biodegradable Poly(ester-urethane) Carriers Exhibiting Controlled Release of Epirubicin. Pharm Res 2017, 34 (4), 780-792.
36. Aluri, R.; Saxena, S.; Joshi, D. C.; Jayakannan, M., Multistimuli-Responsive Amphiphilic Poly(ester-urethane) Nanoassemblies Based on 1-Tyrosine for Intracellular Drug Delivery to Cancer Cells. Biomacromolecules 2018, 19 (6), 2166-2181.
37. Chaffin, K. A.; Chen, X.; McNamara, L.; Bates, F. S.; Hillmyer, M. A., Polyether Urethane Hydrolytic Stability after Exposure to Deoxygenated Water. Macromolecules 2014, 47 (15), 5220-5226.
38. Zeng, M.; Shen, J.; Liu, Y.; Lu, L. Y.; Ding, K.; Fortmann, S. D.; Khan, M.; Wang, J.; Hackett, S. F.; Semenza, G. L.; Campochiaro, P. A., The HIF-1 antagonist acriflavine: visualization in retina and suppression of ocular neovascularization. J Mol Med (Berl) 2017, 95 (4), 417-429.
39. Lee, K.; Zhang, H.; Qian, D. Z.; Rey, S.; Liu, J. O.; Semenza, G. L., Acriflavine inhibits HIF-1 dimerization, tumor growth, and vascularization. Proc Natl Acad Sci USA 2009, 106 (42), 17910-5.
40. Montigaud, Y.; Ucakar, B.; Krishnamachary, B.; Bhujwalla, Z. M.; Feron, O.; Préat, V.; Danhier, F.; Gallez, B.; Danhier, P., Optimized acriflavine-loaded lipid nanocapsules as a safe and effective delivery system to treat breast cancer. Int J Pharm 2018, 551 (1-2), 322-328.
41. Lian, G.; Li, X.; Zhang, L.; Zhang, Y.; Sun, L.; Zhang, X.; Liu, H.; Pang, Y.; Kong, W.; Zhang, T.; Wang, X.; Jiang, C., Macrophage metabolic reprogramming aggravates aortic dissection through the HIF1α-ADAM17 pathway(⋆). EBioMedicine 2019, 49, 291-304.
42. Cheloni, G.; Tanturli, M.; Tusa, I.; Ho DeSouza, N.; Shan, Y.; Gozzini, A.; Mazurier, F.; Rovida, E.; Li, S.; Dello Sbarba, P., Targeting chronic myeloid leukemia stem cells with the hypoxia-inducible factor inhibitor acriflavine. Blood 2017, 130 (5), 655-665.
43. Picheth, G.; Houvenagel, S.; Dejean, C.; Couture, O.; Alves de Freitas, R.; Moine, L.; Tsapis, N., Echogenicity enhancement by end-fluorinated polylactide perfluorohexane nanocapsules: Towards ultrasound-activable nanosystems. Acta Biomater 2017, 64, 313-322.
44. Houvenagel, S.; Moine, L.; Picheth, G.; Dejean, C.; Brulet, A.; Chennevière, A.; Faugeras, V.; Huang, N.; Couture, O.; Tsapis, N., Comb-Like Fluorophilic-Lipophilic-Hydrophilic Polymers for Nanocapsules as Ultrasound Contrast Agents. Biomacromolecules 2018, 19 (8), 3244-3256.
45. Achmad, A.; Yamaguchi, A.; Hanaoka, H.; Tsushima, Y., Thin-Shelled PEGylated Perfluorooctyl Bromide Nanocapsules for Tumor-Targeted Ultrasound Contrast Agent. Contrast media & molecular imaging 2018, 2018, 1725323.
46. Phenix, C. P.; Togtema, M.; Pichardo, S.; Zehbe, I.; Curiel, L., High intensity focused ultrasound technology, its scope and applications in therapy and drug delivery. Journal of pharmacy & pharmaceutical sciences: a publication of the Canadian Society for Pharmaceutical Sciences, Societe canadienne des sciences pharmaceutiques 2014, 17 (1), 136-53.
47. Cui, G.; Wang, J.; Wang, X.; Li, W.; Zhang, X., Preparation and Properties of Narrowly Dispersed Polyurethane Nanocapsules Containing Essential Oil via Phase Inversion Emulsification. Journal of agricultural and food chemistry 2018, 66 (41), 10799-10807.
48. Andrieu, J.; Kotman, N.; Maier, M.; Mailänder, V.; Strauss, W. S.; Weiss, C. K.; Landfester, K., Live monitoring of cargo release from peptide-based hybrid nanocapsules induced by enzyme cleavage. Macromolecular rapid communications 2012, 33 (3), 248-53.
49. Pramanik, S. K.; Sreedharan, S.; Singh, H.; Khan, M.; Tiwari, K.; Shiras, A.; Smythe, C.; Thomas, J. A.; Das, A., Mitochondria Targeting Non-Isocyanate-Based Polyurethane Nanocapsules for Enzyme-Triggered Drug Release. Bioconjug Chem 2018, 29 (11), 3532-3543.
50. Powers, J.; Kremkau, F., Medical ultrasound systems. Interface focus 2011, 1 (4), 477-89.
51. Jensen, J. A., Medical ultrasound imaging. Progress in biophysics and molecular biology 2007, 93 (1-3), 153-65.
52. Sirsi, S. R.; Borden, M. A., State-of-the-art materials for ultrasound-triggered drug delivery. Adv Drug Deliv Rev 2014, 72, 3-14.
53. Xu, J.; Tu, H.; Ao, Z.; Chen, Y.; Danehy, R.; Guo, F., Acoustic disruption of tumor endothelium and on-demand drug delivery for cancer chemotherapy. Nanotechnology 2019, 30 (15), 154001.
54. Rocas, P.; Fernández, Y.; Garcia-Aranda, N.; Foradada, L.; Calvo, P.; Avilés, P.; Guillén, M. J.; Schwartz, S., Jr.; Rocas, J.; Albericio, F.; Abasolo, I., Improved pharmacokinetic profile of lipophilic anti-cancer drugs using αvβ3-targeted polyurethane-polyurea nanoparticles. Nanomedicine 2018, 14 (2), 257-267.
55. da Cruz, L.; Fynes, K.; Georgiadis, O.; Kerby, J.; Luo, Y. H.; Ahmado, A.; Vernon, A.; Daniels, J. T.; Nommiste, B.; Hasan, S. M.; Gooljar, S. B.; Carr, A. F.; Vugler, A.; Ramsden, C. M.; Bictash, M.; Fenster, M.; Steer, J.; Harbinson, T.; Wilbrey, A.; Tufail, A.; Feng, G.; Whitlock, M.; Robson, A. G.; Holder, G. E.; Sagoo, M. S.; Loudon, P. T.; Whiting, P.; Coffey, P. J., Phase 1 clinical study of an embryonic stem cell-derived retinal pigment epithelium patch in age-related macular degeneration. Nat Biotechnol 2018, 36 (4), 328-337.
56. Kamao, H., Preclinical Study of Human Induced Pluripotent Stem Cell-derived Retinal Pigment Epithelium Cell Sheets Transplantation. Nippon Ganka Gakkai zasshi 2016, 120 (11), 754-63.
57. Ambati, J.; Fowler, B. J., Mechanisms of age-related macular degeneration. Neuron 2012, 75 (1), 26-39.
58. Spilsbury, K.; Garrett, K. L.; Shen, W. Y.; Constable, I. J.; Rakoczy, P. E., Overexpression of vascular endothelial growth factor (VEGF) in the retinal pigment epithelium leads to the development of choroidal neovascularization. Am J Pathol 2000, 157 (1), 135-44.
59. Gragoudas, E. S.; Adamis, A. P.; Cunningham, E. T., Jr.; Feinsod, M.; Guyer, D. R., Pegaptanib for neovascular age-related macular degeneration. N Engl J Med 2004, 351 (27), 2805-16.
60. Martin, D. F.; Maguire, M. G.; Ying, G. S.; Grunwald, J. E.; Fine, S. L.; Jaffe, G. J., Ranibizumab and bevacizumab for neovascular age-related macular degeneration. N Engl J Med 2011, 364 (20), 1897-908.
61. Rosenfeld, P. J.; Rich, R. M.; Lalwani, G. A., Ranibizumab: Phase III clinical trial results. Ophthalmology clinics ofNorth America 2006, 19 (3), 361-72.
62. Semeraro, F.; Morescalchi, F.; Duse, S.; Parmeggiani, F.; Gambicorti, E.; Costagliola, C., Aflibercept in wet AMD: specific role and optimal use. Drug design, development and therapy 2013, 7, 711-22.
63. Little, K., J. H. Ma, N. Yang, M. Chen, and H. Xu, Myofibroblasts in macular fibrosis secondary to neovascular age-related macular degeneration—the potential sources and molecular cues for their recruitment and activation. EBioMedicine, 2018. 38: p. 283-291. PMID: 30473378. pmcid: PMC6306402.
64. Yerramothu, P., New Therapies of Neovascular AMD-Beyond Anti-VEGFs. Vision (Basel), 2018. 2(3). PMID: 31735894. pmcid: PMC6835305.
65. Lancaster, L. H., J. A. de Andrade, J. D. Zibrak, M. L. Padilla, C. Albera, S. D. Nathan, M. S. Wijsenbeek, J. L. Stauffer, K. U. Kirchgaessler, and U. Costabel, Pirfenidone safety and adverse event management in idiopathic pulmonaryfibrosis. Eur Respir Rev, 2017. 26(146). PMID: 29212837.
66. Gao, C., X. Cao, L. Huang, Y. Bao, T. Li, Y. Di, L. Wu, and Y. Song, Pirfenidone Alleviates Choroidal Neovascular Fibrosis through TGF-beta Smad Signaling Pathway. J Ophthalmol, 2021. 2021: p. 8846708. PMID: 33628482. pmcid: PMC7889376.
67. Oku, H., T. Shimizu, T. Kawabata, M. Nagira, I. Hikita, A. Ueyama, S. Matsushima, M. Toni, and A. Arimura, Antifibrotic action of pirfenidone and prednisolone: different effects on pulmonary cytokines and growth factors in bleomycin-induced murine pulmonary fibrosis. Eur J Pharmacol, 2008. 590(1-3): p. 400-8. PMID: 18598692.
68. Liu, X., Y. Yang, X. Guo, L. Liu, K. Wu, and M. Yu, The Antiangiogenesis Effect of Pirfenidone in Wound Healing In Vitro. J Ocul Pharmacol Ther, 2017. 33(9): p. 693-703. PMID: 28933986.
69. Gan, D., W. Cheng, L. Ke, A. R. Sun, Q. Jia, J. Chen, J. Lin, J. Li, Z. Xu, and P. Zhang, Repurposing of Pirfenidone (Anti-Pulmonary Fibrosis Drug) for Treatment of Rheumatoid Arthritis. Front Pharmacol, 2021. 12: p. 631891. PMID: 33746759. pmcid: PMC7973213.
70. Bao, Y., L. Huang, X. Huang, C. Gao, Y. Chen, L. Wu, S. Zhu, and Y. Song, Pirfenidone ameliorates the formation of choroidal neovascularization in mice. Mol Med Rep, 2020. 21(5): p. 2162-2170. PMID: 32323767. pmcid: PMC7115199.
71. Jiang, N., M. Ma, Y. Li, T. Su, X. Z. Zhou, L. Ye, Q. Yuan, P. Zhu, Y. Min, W. Shi, X. Xu, J. Lv, and Y. Shao, The role of pirfenidone in alkali burn rat cornea. Int Immunopharmacol, 2018. 64: p. 78-85. PMID: 30153530.
72. Sun, G., X. Lin, H. Zhong, Y. Yang, X. Qiu, C. Ye, K. Wu, and M. Yu, Pharmacokinetics of pirfenidone after topical administration in rabbit eye. Mol Vis, 2011. 17: p. 2191-6. PMID: 21866212. pmcid: PMC3159682.
73. Trivedi, R., E. F. Redente, A. Thakur, D. W. Riches, and U. B. Kompella, Local delivery of biodegradable pirfenidone nanoparticles ameliorates bleomycin-induced pulmonary fibrosis in mice. Nanotechnology, 2012. 23(50): p. 505101. PMID: 23186914.
74. Abnoos, M., M. Mohseni, S. A. J. Mousavi, K. Ashtari, R. Ilka, and B. Mehravi, Chitosan-alginate nano-carrier for transdermal delivery of pirfenidone in idiopathic pulmonary fibrosis. Int J Biol Macromol, 2018. 118(Pt A): p. 1319-1325. PMID: 29715556.
75. Jose, A., P. K. Mandapalli, and V. V. Venuganti, Liposomal hydrogel formulation for transdermal delivery of pirfenidone. J Liposome Res, 2016. 26(2): p. 139-47. PMID: 26114208.
76. Ying, G. S., B. J. Kim, M. G. Maguire, J. Huang, E. Daniel, G. J. Jaffe, J. E. Grunwald, K. J. Blinder, C. J. Flaxel, F. Rahhal, C. Regillo, and D. F. Martin, Sustained visual acuity loss in the comparison of age-related macular degeneration treatments trials. JAMA Ophthalmol, 2014. 132(8): p. 915-21. PMID: 24875610. pmcid: PMC4151260.
77. Taylor, D. J., A. E. Hobby, A. M. Binns, and D. P. Crabb, How does age-related macular degeneration affect real-world visual ability and quality of life? A systematic review. BMJ Open, 2016. 6(12): p. e011504. PMID: 27913556. pmcid: PMC5168634.
78. Mettu, P. S., M. J. Allingham, and S. W. Cousins, Incomplete response to Anti-VEGF therapy in neovascular AMD: Exploring disease mechanisms and therapeutic opportunities. Prog Retin Eye Res, 2020: p. 100906. PMID: 33022379.
79. Gräfe, M. G. O., J. A. van de Kreeke, J. Willemse, B. Braaf, Y. de Jong, H. S. Tan, F. D. Verbraak, and J. F. de Boer, Subretinal Fibrosis Detection Using Polarization Sensitive Optical Coherence Tomography. Transl Vis Sci Technol, 2020. 9(4): p. 13. PMID: 32818100. pmcid: PMC7396173.
80. Luo, X., S. Yang, J. Liang, Y. Zhai, M. Shen, J. Sun, Y. Feng, X. Lu, H. Zhu, F. Wang, and X. Sun, Choroidal pericytes promote subretinal fibrosis after experimental photocoagulation. Dis Model Mech, 2018. 11(4). PMID: 29622551. pmcid: PMC5963858.
81. Yasukawa, T., Y. Tabata, H. Kimura, and Y. Ogura, Recent advances in intraocular drug delivery systems. Recent Pat Drug Deliv Formul, 2011. 5(1): p. 1-10. PMID: 21143129.
82. Kompella, U. B., R. S. Kadam, and V. H. Lee, Recent advances in ophthalmic drug delivery. Ther Deliv, 2010. 1(3): p. 435-456. PMID: 21399724.
83. Dixon, P., T. Ghosh, K. Mondal, A. Konar, A. Chauhan, and S. Hazra, Controlled delivery of pirfenidone through vitamin E-loaded contact lens ameliorates corneal inflammation. Drug Deliv Transl Res, 2018. 8(5): p. 1114-1126. PMID: 29858820.
84. Wu, C., P. W. Or, J. I. T. Chong, K. P. D. IK, C. H. C. Lee, K. Wu, M. Yu, D. C. C. Lam, and Y. Yang, Controllable release of pirfenidone by polyvinyl alcohol film embedded soft contact lenses in vitro and in vivo. Drug Deliv, 2021. 28(1): p. 634-641. PMID: 33779455.
85. Hackett, S. F., J. Fu, Y. C. Kim, H. Tsujinaka, J. Shen, E. S. R. Lima, M. Khan, Z. Hafiz, T. Wang, M. Shin, N. M. Anders, P. He, L. M. Ensign, J. Hanes, and P. A. Campochiaro, Sustained delivery of acriflavine from the suprachoroidal space provides long term suppression of choroidal neovascularization. Biomaterials, 2020. 243: p. 119935. PMID: 32172031. pmcid: PMC7249226.
86. Onwukwe, C., N. Maisha, M. Holland, M. Varley, R. Groynom, D. Hickman, N. Uppal, A. Shoffstall, J. Ustin, and E. Lavik, Engineering Intravenously Administered Nanoparticles to Reduce Infusion Reaction and Stop Bleeding in a Large Animal Model of Trauma. Bioconjug Chem, 2018. 29(7): p. 2436-2447. PMID: 29965731. pmcid: PMC6830447.
87. Maisha, N., T. Coombs, and E. Lavik, Development of a Sensitive Assay to Screen Nanoparticles in Vitro for Complement Activation. ACS Biomaterials Science & Engineering, 2020. 6(9): p. 4903-4915.
88. Rohrer, B., Q. Long, B. Coughlin, R. B. Wilson, Y. Huang, F. Qiao, P. H. Tang, K. Kunchithapautham, G. S. Gilkeson, and S. Tomlinson, A targeted inhibitor of the alternative complement pathway reduces angiogenesis in a mouse model of age-related macular degeneration. Invest Ophthalmol Vis Sci, 2009. 50(7): p. 3056-64. PMID: 19264882.

1. Polyurethane nanocapsules comprising a substantially spherical shell of polyurethane surrounding a core, wherein the shell comprising the polyurethane further comprises at least one molecule to be encapsulated.

2. The polyurethane nanocapsules of claim 1, wherein the at least one molecule is homogeneously or heterogeneously distributed throughout the substantially spherical shell of polyurethane.

3. The polyurethane nanocapsules of claim 1, wherein the at least one molecule is exposed on an outside surface of the shell, exposed on an inside surface of the shell, and/o encapsulated within the shell between the outside surface and the inside surface.

4. The polyurethane nanocapsules of claim 1, wherein the core comprises at least one of air, a hydrophilic liquid, a hydrophobic liquid, or a contrast agent.

5. The polyurethane nanocapsules of claim 1, wherein the molecule to be encapsulated comprises a species selected from the group consisting of acriflavine, pirfenidone, 4-hydroxy-TEMPO, growth factors, AG1478, methotrexate, and antibiotic agents.

6. The polyurethane nanocapsules of claim 1, having an effective mean diameter of about 50 to about 900 nm.

7. The polyurethane nanocapsules of claim 1, wherein the nanocapsules can be lyophilized and resuspended.

8. The polyurethane nanocapsules of claim 1, wherein the polyurethane nanocapsule undergoes long-term passive delivery of the molecule into an environment in proximity of the nanocapsule.

9. The polyurethane nanocapsules of claim 1, wherein the polyurethane nanocapsule can be triggered using ultrasound to release the molecule, on-demand, into the environment in proximity of the nanocapsule.

10. A method of making the polyurethane nanocapsules of claim 1, said method comprising:
dissolving surfactant in water and hexadecane to form a mixture;
stirring or sonicating the mixture at temperature in a range from about 35-45° C.;
adding isophorone diisocyanate (IPDI) to the stirred mixture comprising the water, surfactant, and hexadecane to form a solution;
sonicating the solution to form an emulsion;
adding a hydroxy-containing compound to the emulsion, with continued sonication; and
reacting the IPDI and the hydroxy-containing compound, with stirring, to form the polyurethane nanocapsules encapsulating the at least one molecule,
wherein the molecule to be encapsulated is added with the IPDI or the hydroxy-containing compound, depending on the solubility of the molecule to be encapsulated.

11. A method of inhibiting neovascularization in an eye of a subject in need thereof, said method comprising administering a long-term delivery system comprising the polyurethane nanocapsules of claim 1 to the eye of said subject, wherein the at least one molecule to be encapsulated comprises acriflavine.

12. The method of claim 11, wherein the polyurethane nanocapsules can passively deliver the acriflavine and/or they can be repeatedly triggered to release highly controlled and reproducible amounts of acriflavine in response to an amount of ultrasound energy applied to the nanocapsules.

13. A method of substantially reducing or eliminating fibrosis in an eye of a subject in need thereof, said method comprising administering a long-term delivery system comprising the polyurethane nanocapsules of claim 1 to the eye of said subject, wherein the at least one molecule to be encapsulated comprises pirfenidone.

14. The method of claim 13, wherein the polyurethane nanocapsules can passively deliver the pirfenidone and/or they can be repeatedly triggered to release highly controlled and reproducible amounts of pirfenidone in response to an amount of ultrasound energy applied to the nanocapsules.

15. A method of inhibiting neovascularization and substantially reducing or eliminating fibrosis in an eye of a subject in need thereof, said method comprising administering a combination therapy or dual delivery system comprising the polyurethane nanocapsules of claim 1 to the eye of said subject, wherein some portion of the polyurethane nanocapsules comprise encapsulated acriflavine and the remaining portion of the polyurethane nanocapsules comprise encapsulated pirfenidone.

16. The method of claim 15, wherein the polyurethane nanocapsules can passively deliver the pirfenidone and acriflavine and/or they can be repeatedly triggered to release highly controlled and reproducible amounts of pirfenidone and acriflavine in response to an amount of ultrasound energy applied to the nanocapsules.

17. A method of treating wet-AMD, or preserving vision, in an eye of a subject in need thereof, said method comprising administering a long-term delivery system comprising the polyurethane nanocapsules of claim 1 to the eye of said subject, wherein the at least one molecule to be encapsulated is selected from acriflavine, pirfenidone, or both acriflavine and pirfenidone.

18. The method of claim 17, wherein the polyurethane nanocapsules can passively deliver the pirfenidone and acriflavine and/or they can be repeatedly triggered to release highly controlled and reproducible amounts of pirfenidone and acriflavine in response to an amount of ultrasound energy applied to the nanocapsules.

19. The method of claim 11, wherein the polyurethane nanocapsules can be administered intravitreally, suprachoroidally, or subcunjunctivally.
